(19) United States
(12) Patent Application Publication
(10) Pub. No.: US 2019/0002480 A1
(43) Pub. Date: Jan. 3, 2019

(54) Silane-Containing Heterocyclic Compounds and Methods of Use Thereof for the Treatment of Viral Diseases

(71) Applicants: Joseph A. KOZLOWSKI, Kenilworth, NJ (US); Wensheng YU, Rahway, NJ (US); Merck Sharp & Dohme Corp., Rahway, NJ (US)

(72) Inventors: Joseph A. Kozlowski, Princeton, NJ (US); Wensheng Yu, Edison, NJ (US)

(73) Assignee: Merck Sharp & Dohme Corp., Rahway, NJ (US)

(21) Appl. No.: 16/064,268

(22) PCT Filed: Dec. 16, 2016
(86) PCT No.: PCT/US2016/067071

§ 371 Date: Jun. 20, 2018

Related U.S. Application Data
(60) Provisional application No. 62/270,271, filed on Dec. 21, 2015.

(51) Int. Cl. C07F 7/08; C07D 498/04; A61P 31/14

(52) U.S. Cl. CPC C07F 7/0816; C07D 498/04; A61K 31/7056; A61K 2300/00; A61P 31/14

(57) ABSTRACT

The present invention relates to novel Silane-Containing Heterocyclic Compounds of Formula (I): and pharmaceutically acceptable salts thereof, wherein A, A′, B, B′, R1 R2 and R3 are as defined herein. The present invention also relates to compositions comprising at least one Silane-Containing Heterocyclic Compound, and methods of using the Silane-Containing Heterocyclic Compounds for treating or preventing HCV infection in a patient.

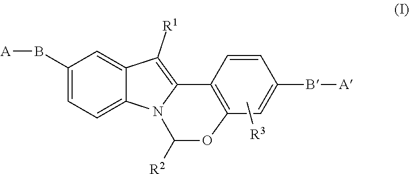

### FIELD OF THE INVENTION

The present invention relates to novel Silane-Containing Heterocyclic Compounds, compositions comprising at least one Silane-Containing Heterocyclic Compound, and methods of using the Silane-Containing Heterocyclic Compounds for treating or preventing HCV infection in a patient.

### BACKGROUND OF THE INVENTION

Hepatitis C virus (HCV) is a major human pathogen. A substantial fraction of these HCV-infected individuals develop serious progressive liver disease, including cirrhosis and hepatocellular carcinoma, which are often fatal.

Recent attention has been focused toward the identification of inhibitors of HCV NS5A. HCV NS5A is a 447 amino acid phosphoprotein which lacks a defined enzymatic function. It runs as 56 kd and 58 kd bands on gels depending on phosphorylation state (Tanji, et al. J. Virol. 69:3980-3986 (1995)). HCV NS5A resides in replication complex and may be responsible for the switch from replication of RNA to production of infectious virus (Huang, Y, et al., Virology 364:1-9 (2007)).

Multicyclic HCV NS5A inhibitors have been reported. See U.S. Patent Publication Nos. US20080311075, US20080044379, US20080050336, US20080044380, US20090202483 and US2009020478. HCV NS5A inhibitors having fused tricyclic moieties are disclosed in PCT International Patent Publication Nos. WO 10/065681, WO 10/065668, and WO 10/065674.

Other HCV NS5A inhibitors and their use for reducing viral load in HCV infected humans have been described in U.S. Patent Publication No. US20060276511.

### SUMMARY OF THE INVENTION

In one aspect, the present invention provides Compounds of Formula

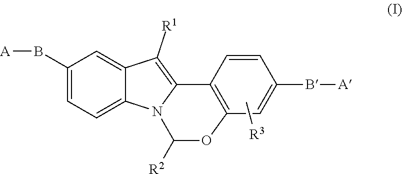

or a pharmaceutically acceptable salt thereof, wherein:

A is selected from 4 to 7-membered monocyclic heterocycloalkyl or R¹¹, wherein said 4 to 7-membered monocyclic heterocycloalkyl group and said R¹¹ group is substituted on a ring nitrogen atoms with R⁴, and optionally further substituted on one or more ring carbon atoms with R⁵, such that two R⁵ groups on the same ring carbon atom, together with the carbon atom to which they are attached, can join to form a spirocyclic C₃-C₇ cycloalkyl group or a spirocyclic 4 to 7-membered monocyclic heterocycloalkyl group; or two R⁵ groups attached to the same A ring, together with the carbon atoms to which they are attached, can join to form a fused C₃-C₇ cycloalkyl group, a bridged C₃-C₇ cycloalkyl group or a fused 4 to 7-membered monocyclic heterocycloalkyl group;

A′ is selected from 4 to 7-membered monocyclic heterocycloalkyl or R¹¹, wherein said 4 to 7-membered monocyclic heterocycloalkyl group and said R¹¹ group is substituted on a ring nitrogen atoms with R⁴, and optionally further substituted on one or more ring carbon atoms with R⁵, such that two R⁵ groups on the same ring carbon atom, together with the carbon atom to which they are attached, can join to form a spirocyclic C₃-C₇ cycloalkyl group or a spirocyclic 4 to 7-membered monocyclic heterocycloalkyl group; or two R⁵ groups attached to the same A ring, together with the carbon atoms to which they are attached, can join to form a fused C₃-C₇ cycloalkyl group, a bridged C₃-C₇ cycloalkyl group or a fused 4 to 7-membered monocyclic heterocycloalkyl group;

B is:

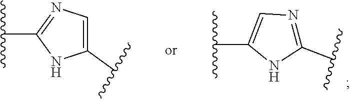 [SMILES: *c1cnc(*)[nH]1 ; *c1cnc(*)[nH]1]

B′ is:

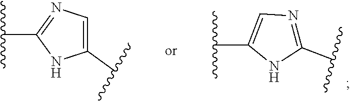 [SMILES: *c1cnc(*)[nH]1 ; *c1cnc(*)[nH]1]

R¹ is selected from H, C₁-C₆ alkyl, C₃-C₇ cycloalkyl, 4 to 7-membered heteroaryl, phenyl and halo;

R² is selected from 5 or 6-membered monocyclic heteroaryl and 9 or 10-membered bicyclic heteroaryl, wherein said 5 or 6-membered monocyclic heteroaryl group and said 9 or 10-membered bicyclic heteroaryl group each can be optionally substituted on one or more ring carbon atoms with R⁶;

R³ represents up to 3 optional phenyl group substituents, each independently selected from halo, —CN, C₁-C₆ alkyl, C₁-C₆ haloalkyl, C₃-C₇ cycloalkyl, 4 to 6-membered monocyclic heterocycloalkyl, 5 or 6-membered monocyclic heteroaryl, C₆-C₁₀ aryl, benzyl and —O—(C₁-C₆ alkyl), wherein said C₃-C₇ cycloalkyl group, said 4 to 6-membered monocyclic heterocycloalkyl group, said 5 or 6-membered monocyclic heteroaryl group, said C₆-C₁₀ aryl group, or the phenyl moiety of said benzyl group can be optionally substituted with up to 3 groups, which can be the same or different, and are selected from halo, —CN, C₁-C₆ alkyl, C₁-C₆ haloalkyl, —O—C₁-C₆ alkyl, —(C₁-C₆ alkylene)-O—C₁-C₆ alkyl and —O—(C₁-C₆ haloalkyl);

each occurrence of R⁴ is independently:

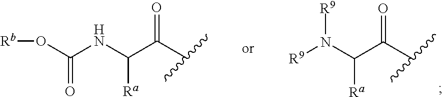

R^{a} is selected from C₁-C₆ alkyl, C₁-C₆ haloalkyl, C₁-C₆ silylalkyl, C₃-C₇ cycloalkyl, 4- to 7-membered monocyclic heterocycloalkyl, phenyl and 5 or 6-membered monocyclic heteroaryl, wherein said C₃-C₇ cycloalkyl group, said 4 to 7-membered monocyclic heterocycloalkyl group, said phenyl group, and said 5 or 6-membered monocyclic heteroaryl group can each be optionally substituted on one or more ring carbon atoms with R⁶;

R^{b} is selected from H, C₁-C₆ alkyl, C₁-C₆ haloalkyl, C₁-C₆ silylalkyl, C₃-C₇ cycloalkyl, 4- to 7-membered monocyclic heterocycloalkyl, phenyl and 5 or 6-membered monocyclic heteroaryl;

each occurrence of R⁵ is independently selected from C₁-C₆ alkyl, C₁-C₆ haloalkyl, C₃-C₇ cycloalkyl, 4 to 7-membered monocyclic heterocycloalkyl, phenyl, 5 or 6-membered monocyclic heteroaryl, halo, —CN, —OR⁸, —N(R⁷)₂, —C(O)R¹⁰, —C(O)OR⁸, —C(O)N(R⁸)₂, —NHC(O)R⁸, —NHC(O)NHR⁸, —NHC(O)OR⁸, —OC(O)R⁸, —SR⁸, —S(O)₂R⁸ and Si(R¹⁰)₃, wherein said C₃-C₇ cycloalkyl group, said 4 to 7-membered monocyclic heterocycloalkyl group, said phenyl group, and said 5 or 6-membered monocyclic heteroaryl group can each be optionally substituted on one or more ring carbon atoms with R⁶;

each occurrence of R⁶ is independently selected from halo, —CN, C₁-C₆ alkyl, C₃-C₇ cycloalkyl, C₁-C₆ haloalkyl, —O—(C₁-C₆ haloalkyl), C₂-C₆ alkynyl, C₁-C₆ hydroxyalkyl, —(C₁-C₆ alkylene)ₘ-O—C₁-C₆ alkyl, —N(R⁷)₂, C₆-C₁₀ aryl, —(C₁-C₆ alkylene)ₘ-(C₃-C₇ cycloalkyl), —O—(C₆-C₁₀ aryl), 4 to 7-membered monocyclic heterocycloalkyl, 5 or 6-membered monocyclic heteroaryl, —O-(5 or 6-membered monocyclic heteroaryl), 8 to 10-membered bicyclic heteroaryl and —O-(8 to 10-membered bicyclic heteroaryl), wherein said C₆-C₁₀ aryl group, said C₃-C₇ cycloalkyl group, said 4 to 7-membered monocyclic heterocycloalkyl group, said 5 or 6-membered monocyclic heteroaryl group and said 8 to 10-membered bicyclic heteroaryl group can be optionally substituted with up to 3 groups, each independently selected from halo, hydroxy, C₁-C₆ alkyl, C₁-C₆ haloalkyl and —O—C₁-C₆ alkyl, and wherein said C₆-C₁₀ aryl group, said 5 or 6-membered monocyclic heteroaryl group and said 8 to 10-membered bicyclic heteroaryl group, can be optionally fused with a C₃-C₆ cycloalkyl group;

each occurrence of R⁷ is independently selected from H, C₁-C₆ alkyl, or C₃-C₇ cycloalkyl;

each occurrence of R⁸ is independently selected from H, C₁-C₆ alkyl, C₁-C₆ haloalkyl, —C₁-C₆ alkylene-OC(O)(C₁-C₆ alkyl), C₁-C₆ hydroxyalkyl, C₃-C₇ cycloalkyl, 4 to 7-membered monocyclic heterocycloalkyl, phenyl and 5 or 6-membered monocyclic heteroaryl, wherein said C₃-C₇ cycloalkyl group, said 4- to 7-membered monocyclic heterocycloalkyl group, said phenyl group and said 5 or 6-membered monocyclic heteroaryl group can be optionally and independently substituted with up to three groups independently selected from —OH, halo, C₁-C₆ alkyl, C₁-C₆ haloalkyl, —NH(C₁-C₆ alkyl) and —N(C₁-C₆ alkyl)₂;

each occurrence of R⁹ is independently selected from H, C₁-C₆ alkyl, or C₃-C₇ cycloalkyl, or two R⁹ groups, together with the common N to which they are attached, can join to form a 3 to 7 membered heterocycloalkyl group;

each occurrence of R¹⁰ is independently selected from C₁-C₆ alkyl, C₃-C₇ cycloalkyl, 4- to 7-membered monocyclic heterocycloalkyl, phenyl, 5 or 6-membered monocyclic heteroaryl, C₁-C₆ haloalkyl, —CN and —OR³, wherein said C₃-C₇ cycloalkyl group, said 4 to 7-membered monocyclic heterocycloalkyl group, said phenyl group, and said 5 or 6-membered monocyclic heteroaryl group can each be optionally substituted on one or more ring carbon atoms with R⁶, or optionally, two R¹⁰ groups, together with the common silicon atom to which they are attached, can optionally join to form a 4- to 7-membered silyl-containing monocyclic heterocycloalkyl ring;

each occurrence of R¹¹ is independently selected from monocyclic 5- to 7-membered silylheterocycloalkyl ring and a bicyclic 7- to 11-membered silylheterocycloalkyl ring wherein said silylheterocycloalkyl rings contain as heteroatom ring members:

(i) one —Si(R¹⁰)₂—;

(ii) one —N(R⁴)—; and

(iii) one optional and additional heteroatom ring member selected
from the group consisting of nitrogen, oxygen and sulfur, and wherein an R¹¹ group can be optionally and independently substituted on one or two ring carbon atoms with R⁵; and

each occurrence of m is independently 0 or 1;

wherein at least one of A and A′ is R¹¹.

The Compounds of Formula (I) (also referred to herein as the “Silane-Containing Heterocyclic Compounds”) and pharmaceutically acceptable salts thereof can be useful, for example, for inhibiting HCV viral replication or replicon activity, and have the potential for treating or preventing HCV infection in a patient. Without being bound by any specific theory, it is believed that the Silane-Containing Heterocyclic Compounds inhibit HCV viral replication by inhibiting HCV NS5A.

Accordingly, the present invention provides methods for treating or preventing HCV infection in a patient, comprising administering to the patient an effective amount of at least one Silane-Containing Heterocyclic Compound.

The details of the invention are set forth in the accompanying detailed description below.

Although any methods and materials similar to those described herein can be used in the practice or testing of the present invention, illustrative methods and materials are now described. Other embodiments, aspects and features of the present invention are either further described in or will be apparent from the ensuing description, examples and appended claims.

### DETAILED DESCRIPTION OF THE INVENTION

The present invention relates to novel Silane-Containing Heterocyclic Compounds, compositions comprising at least one Silane-Containing Heterocyclic Compound, and methods of using the Silane-Containing Heterocyclic Compounds for treating or preventing HCV infection in a patient.

### Definitions and Abbreviations

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “-O-alkyl,” etc. . . . .

As used herein, and throughout this disclosure, the following terms, unless otherwise indicated, shall be understood to have the following meanings:

A “patient” is a human or non-human mammal. In one embodiment, a patient is a human. In another embodiment, a patient is a chimpanzee.

The term “effective amount” as used herein, refers to an amount of Silane-Containing Heterocyclic Compound and/or an additional therapeutic agent, or a composition thereof that is effective in producing the desired therapeutic, ameliorative, inhibitory or preventative effect when administered to a patient suffering from a viral infection or virus-related disorder. In the combination therapies of the present invention, an effective amount can refer to each individual agent or to the combination as a whole, wherein the amounts of all agents administered are together effective, but wherein the component agent of the combination may not be present individually in an effective amount.

The term “preventing,” as used herein with respect to an HCV viral infection or HCV-virus related disorder, refers to reducing the likelihood of HCV infection.

The term “alkyl,” as used herein, refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 12 carbon atoms. In different embodiments, an alkyl group contains from 1 to 6 carbon atoms (C₁-C₆ alkyl) or from about 1 to about 4 carbon atoms (C₁-C₄ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkyl group is linear. In another embodiment, an alkyl group is branched. Unless otherwise indicated, an alkyl group is unsubstituted.

The term “alkenyl,” as used herein, refers to an aliphatic hydrocarbon group containing at least one carbon-carbon double bond and having one of its hydrogen atoms replaced with a bond. An alkenyl group may be straight or branched and contain from about 2 to about 15 carbon atoms. In one embodiment, an alkenyl group contains from about 2 to about 12 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)— cycloalkyl, —C(O)OH and —C(O)O-alkyl. The term “C₂-C₆ alkenyl” refers to an alkenyl group having from 2 to 6 carbon atoms. Unless otherwise indicated, an alkenyl group is unsubstituted.

The term “alkynyl,” as used herein, refers to an aliphatic hydrocarbon group containing at least one carbon-carbon triple bond and having one of its hydrogen atoms replaced with a bond. An alkynyl group may be straight or branched and contain from about 2 to about 15 carbon atoms. In one embodiment, an alkynyl group contains from about 2 to about 12 carbon atoms. In another embodiment, an alkynyl group contains from about 2 to about 6 carbon atoms. Non-limiting examples of alkynyl groups include ethynyl, propynyl, 2-butynyl and 3-methylbutynyl. An alkynyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. The term “C₂-C₆ alkynyl” refers to an alkynyl group having from 2 to 6 carbon atoms. Unless otherwise indicated, an alkynyl group is unsubstituted.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂—, —CH(CH₃)— and —CH₂CH(CH₃)CH₂—. In one embodiment, an alkylene group has from 1 to about 6 carbon atoms. In another embodiment, an alkylene group is branched. In another embodiment, an alkylene group is linear. In one embodiment, an alkylene group is —CH₂—. The term “C₁-C₆ alkylene” refers to an alkylene group having from 1 to 6 carbon atoms.

The term “aryl,” as used herein, refers to an aromatic monocyclic or multicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different, and are as defined herein below. In one embodiment, an aryl group can be optionally fused to a cycloalkyl or cycloalkanoyl group. Non-limiting examples of aryl groups include phenyl and naphthyl. In one embodiment, an aryl group is phenyl. Unless otherwise indicated, an aryl group is unsubstituted.

The term “arylene,” as used herein, refers to a bivalent group derived from an aryl group, as defined above, by removal of a hydrogen atom from a ring carbon of an aryl group. An arylene group can be derived from a monocyclic or multicyclic ring system comprising from about 6 to about 14 carbon atoms. In one embodiment, an arylene group contains from about 6 to about 10 carbon atoms. In another embodiment, an arylene group is a naphthylene group. In another embodiment, an arylene group is a phenylene group. An arylene group can be optionally substituted with one or more “ring system substituents” which may be the same or different, and are as defined herein below. An arylene group is divalent and either available bond on an arylene group can connect to either group flanking the arylene group. For example, the group “A-arylene-B,” wherein the arylene group is:

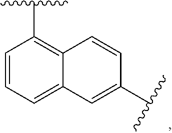 [SMILES: *c1ccc2c(*)cccc2c1]

is understood to represent both:

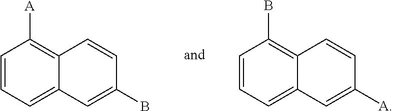

In one embodiment, an arylene group can be optionally fused to a cycloalkyl or cycloalkanoyl group. Non-limiting examples of arylene groups include phenylene and naphthalene. In one embodiment, an arylene group is unsubstituted. In another embodiment, an arylene group is:

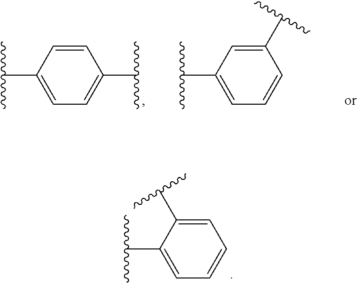

Unless otherwise indicated, an arylene group is unsubstituted.

The term “cycloalkyl,” as used herein, refers to a saturated or unsaturated non-aromatic mono- or multicyclic ring system comprising from about 3 to about 10 ring carbon atoms. In one embodiment, a cycloalkyl contains from about 5 to about 10 ring carbon atoms. In another embodiment, a cycloalkyl contains from about 3 to about 7 ring atoms. In another embodiment, a cycloalkyl contains from about 5 to about 6 ring atoms. The term “cycloalkyl” also encompasses a cycloalkyl group, as defined above, which is fused to an aryl (e.g., benzene) or heteroaryl ring. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl. A cycloalkyl group can be optionally substituted with one or more “ring system substituents” which may be the same or different, and are as defined herein below. In one embodiment, a cycloalkyl group is unsubstituted. The term “3 to 6-membered cycloalkyl” refers to a cycloalkyl group having from 3 to 6 ring carbon atoms. Unless otherwise indicated, a cycloalkyl group is unsubstituted. A ring carbon atom of a cycloalkyl group may be functionalized as a carbonyl group. An illustrative example of such a cycloalkyl group (also referred to herein as a “cycloalkanoyl” group) includes, but is not limited to, cyclobutanoyl:

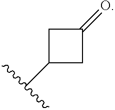 [SMILES: *C1CC(=O)C1]

The term “cycloalkenyl,” as used herein, refers to a non-aromatic mono- or multicyclic ring system comprising from about 4 to about 10 ring carbon atoms and containing at least one endocyclic double bond. In one embodiment, a cycloalkenyl contains from about 4 to about 7 ring carbon atoms. In another embodiment, a cycloalkenyl contains 5 or 6 ring atoms. Non-limiting examples of monocyclic cycloalkenyls include cyclopentenyl, cyclohexenyl, cyclohepta-1,3-dienyl, and the like. A cycloalkenyl group can be optionally substituted with one or more “ring system substituents” which may be the same or different, and are as defined herein below. A ring carbon atom of a cycloalkyl group may be functionalized as a carbonyl group. In one embodiment, a cycloalkenyl group is cyclopentenyl. In another embodiment, a cycloalkenyl group is cyclohexenyl. The term “4 to 6-membered cycloalkenyl” refers to a cycloalkenyl group having from 4 to 6 ring carbon atoms. Unless otherwise indicated, a cycloalkenyl group is unsubstituted.

The term “halo,” as used herein, means —F, —Cl, —Br or —I.

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from 1 to 3 F atoms. Non-limiting examples of haloalkyl groups include —CH₂F, —CHF₂, —CF₃, —CH₂Cl and —CCl₃. The term “C₁-C₆ haloalkyl” refers to a haloalkyl group having from 1 to 6 carbon atoms.

The term “hydroxyalkyl,” as used herein, refers to an alkyl group as defined above, wherein one or more of the alkyl group's hydrogen atoms has been replaced with an —OH group. In one embodiment, a hydroxyalkyl group has from 1 to 6 carbon atoms. Non-limiting examples of hydroxyalkyl groups include —CH₂OH, —CH₂CH₂OH, —CH₂CH₂CH₂OH and —CH₂CH(OH)CH₃. The term “C₁-C₆ hydroxyalkyl” refers to a hydroxyalkyl group having from 1 to 6 carbon atoms.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. In another embodiment, a heteroaryl group is bicyclic and had 9 or 10 ring atoms. A heteroaryl group can be optionally substituted by one or more “ring system substituents” which may be the same or different, and are as defined herein below. A heteroaryl group is joined via a ring carbon atom, and any nitrogen atom of a heteroaryl can be optionally oxidized to the corresponding N-oxide. The term “heteroaryl” also encompasses a heteroaryl group, as defined above, which is fused to a benzene ring. Non-limiting examples of heteroaryls include pyridyl, pyrazinyl, furanyl, thienyl, pyrimidinyl, pyridone (including N-substituted pyridones), isoxazolyl, isothiazolyl, oxazolyl, oxadiazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, benzimidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like, and all isomeric forms thereof. The term “heteroaryl” also refers to partially saturated heteroaryl moieties such as, for example, tetrahydroisoquinolyl, tetrahydroquinolyl and the like. In one embodiment, a heteroaryl group is a 5-membered heteroaryl. In another embodiment, a heteroaryl group is a 6-membered heteroaryl. In another embodiment, a heteroaryl group comprises a 5- to 6-membered heteroaryl group fused to a benzene ring. Unless otherwise indicated, a heteroaryl group is unsubstituted.

The term “heteroarylene,” as used herein, refers to a bivalent group derived from an heteroaryl group, as defined above, by removal of a hydrogen atom from a ring carbon or ring heteroatom of a heteroaryl group. A heteroarylene group can be derived from a monocyclic or multicyclic ring system comprising about 5 to about 14 ring atoms, wherein from 1 to 4 of the ring atoms are each independently O, N or S and the remaining ring atoms are carbon atoms. A heteroarylene group can be optionally substituted by one or more “ring system substituents” which may be the same or different, and are as defined herein below. A heteroarylene group is joined via a ring carbon atom or by a nitrogen atom with an open valence, and any nitrogen atom of a heteroarylene can be optionally oxidized to the corresponding N-oxide. The term “heteroarylene” also encompasses a heteroarylene group, as defined above, which is fused to a benzene ring. Non-limiting examples of heteroarylenes include pyridylene, pyrazinylene, furanylene, thienylene, pyrimidinylene, pyridonylene (including those derived from N-substituted pyridonyls), isoxazolylene, isothiazolylene, oxazolylene, oxadiazolylene, thiazolylene, pyrazolylene, thiophenylene, furazanylene, pyrrolylene, triazolylene, 1,2,4-thiadiazolylene, pyrazinylene, pyridazinylene, quinoxalinylene, phthalazinylene, oxindolylene, imidazo[1,2-a]pyridinylene, imidazo[2,1-b]thiazolylene, benzofurazanylene, indolylene, azaindolylene, benzimidazolylene, benzothienylene, quinolinylene, imidazolylene, benzimidazolylene, thienopyridylene, quinazolinylene, thienopyrimidylene, pyrrolopyridylene, imidazopyridylene, isoquinolinylene, benzoazaindolylene, 1,2,4-triazinylene, benzothiazolylene and the like, and all isomeric forms thereof. The term “heteroarylene” also refers to partially saturated heteroarylene moieties such as, for example, tetrahydroisoquinolylene, tetrahydroquinolylene, and the like. A heteroarylene group is divalent and either available bond on a heteroarylene ring can connect to either group flanking the heteroarylene group. For example, the group “A-heteroarylene-B,” wherein the heteroarylene group is:

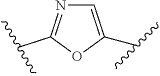 [SMILES: *c1cnc(*)o1]

is understood to represent both:

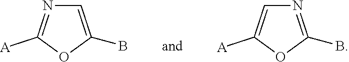

In one embodiment, a heteroarylene group is a monocyclic heteroarylene group or a bicyclic heteroarylene group. In another embodiment, a heteroarylene group is a monocyclic heteroarylene group. In another embodiment, a heteroarylene group is a bicyclic heteroarylene group. In still another embodiment, a heteroarylene group has from about 5 to about 10 ring atoms. In another embodiment, a heteroarylene group is monocyclic and has 5 or 6 ring atoms. In another embodiment, a heteroarylene group is bicyclic and has 9 or 10 ring atoms. In another embodiment, a heteroarylene group is a 5-membered monocyclic heteroarylene. In another embodiment, a heteroarylene group is a 6-membered monocyclic heteroarylene. In another embodiment, a bicyclic heteroarylene group comprises a 5 or 6-membered monocyclic heteroarylene group fused to a benzene ring. Unless otherwise indicated, a heteroarylene group is unsubstituted.

The term “heterocycloalkyl,” as used herein, refers to a non-aromatic saturated monocyclic or multicyclic ring system comprising 3 to about 11 ring atoms, wherein from 1 to 4 of the ring atoms are independently O, S, N or Si, and the remainder of the ring atoms are carbon atoms. A heterocycloalkyl group can be joined via a ring carbon, ring silicon atom or ring nitrogen atom. In one embodiment, a heterocycloalkyl group is monocyclic and has from about 3 to about 7 ring atoms. In another embodiment, a heterocycloalkyl group is monocyclic has from about 4 to about 7 ring atoms. In another embodiment, a heterocycloalkyl group is bicyclic and has from about 7 to about 11 ring atoms. In still another embodiment, a heterocycloalkyl group is monocyclic and has 5 or 6 ring atoms. In one embodiment, a heterocycloalkyl group is monocyclic. In another embodiment, a heterocycloalkyl group is bicyclic. There are no adjacent oxygen and/or sulfur atoms present in the ring system. Any —NH group in a heterocycloalkyl ring may exist protected such as, for example, as an —N(BOC), —N(Cbz), —N(Tos) group and the like; such protected heterocycloalkyl groups are considered part of this invention. The term “heterocycloalkyl” also encompasses a heterocycloalkyl group, as defined above, which is fused to an aryl (e.g., benzene) or heteroaryl ring. A heterocycloalkyl group can be optionally substituted by one or more “ring system substituents” which may be the same or different, and are as defined herein below. The nitrogen or sulfur atom of the heterocycloalkyl can be optionally oxidized to the corresponding N-oxide, S-oxide or S,S-dioxide. Non-limiting examples of monocyclic heterocycloalkyl rings include oxetanyl, piperidyl, pyrrolidinyl, piperazinyl, morpholinyl, thiomorpholinyl, thiazolidinyl, 1,4-dioxanyl, tetrahydrofuranyl, tetrahydrothiophenyl, delta-lactam, delta-lactone, silacyclopentane, silapyrrolidine and the like, and all isomers thereof. Non-limiting illustrative examples of a silyl-containing heterocycloalkyl group include:

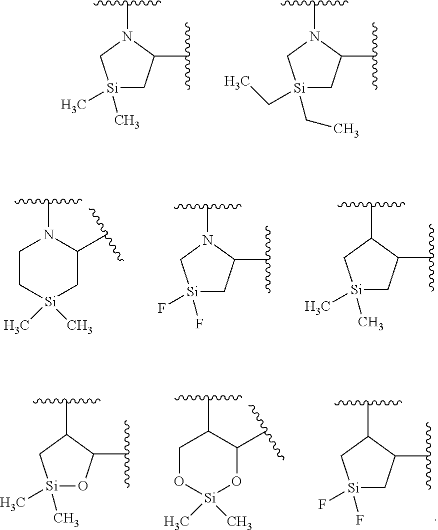

A ring carbon atom of a heterocycloalkyl group may be functionalized as a carbonyl group. An illustrative example of such a heterocycloalkyl group is:

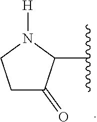 [SMILES: *C1NCCC1=O]

In one embodiment, a heterocycloalkyl group is a 5-membered monocyclic heterocycloalkyl. In another embodiment, a heterocycloalkyl group is a 6-membered monocyclic heterocycloalkyl. The term “3 to 6-membered monocyclic cycloalkyl” refers to a monocyclic heterocycloalkyl group having from 3 to 6 ring atoms. The term “4 to 6-membered monocyclic cycloalkyl” refers to a monocyclic heterocycloalkyl group having from 4 to 6 ring atoms. The term “7 to 11-membered bicyclic heterocycloalkyl” refers to a bicyclic heterocycloalkyl group having from 7 to 11 ring atoms. Unless otherwise indicated, an heterocycloalkyl group is unsubstituted.

The term “heterocycloalkenyl,” as used herein, refers to a heterocycloalkyl group, as defined above, wherein the heterocycloalkyl group contains from 4 to 10 ring atoms, and at least one endocyclic carbon-carbon or carbon-nitrogen double bond. A heterocycloalkenyl group can be joined via a ring carbon or ring nitrogen atom. In one embodiment, a heterocycloalkenyl group has from 4 to 6 ring atoms. In another embodiment, a heterocycloalkenyl group is monocyclic and has 5 or 6 ring atoms. In another embodiment, a heterocycloalkenyl group is bicyclic. A heterocycloalkenyl group can optionally substituted by one or more ring system substituents, wherein “ring system substituent” is as defined above. The nitrogen or sulfur atom of the heterocycloalkenyl can be optionally oxidized to the corresponding N-oxide, S-oxide or S,S-dioxide. Non-limiting examples of heterocycloalkenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluoro-substituted dihydrofuranyl, 7-oxabicyclo[2.2.1]heptenyl, dihydrothiophenyl, dihydrothiopyranyl, and the like and the like. A ring carbon atom of a heterocycloalkenyl group may be functionalized as a carbonyl group. In one embodiment, a heterocycloalkenyl group is a 5-membered heterocycloalkenyl. In another embodiment, a heterocycloalkenyl group is a 6-membered heterocycloalkenyl. The term “4 to 6-membered heterocycloalkenyl” refers to a heterocycloalkenyl group having from 4 to 6 ring atoms. Unless otherwise indicated, a heterocycloalkenyl group is unsubstituted.

Examples of “ring system substituents” include, but are not limited to, alkyl, alkenyl, alkynyl, aryl, heteroaryl, -alkylene-aryl, -arylene-alkyl, -alkylene-heteroaryl, -alkenylene-heteroaryl, -alkynylene-heteroaryl, —OH, hydroxyalkyl, haloalkyl, —O-alkyl, —O-haloalkyl, -alkylene-O-alkyl, —O-aryl, —O-alkylene-aryl, acyl, —C(O)-aryl, halo, —NO₂, —CN, —SF₅, —C(O)OH, —C(O)O-alkyl, —C(O)O-aryl, —C(O)O— alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, —Si(alkyl)₂, —Si(aryl)₂, —Si(heteroaryl)₂, —Si(alkyl)(aryl), —Si(alkyl)(cycloalkyl), —Si(alkyl)(heteroaryl), cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), —N(Y₁)(Y₂), -alkylene-N(Y₁)(Y₂), —C(O)N(Y₁)(Y₂) and —S(O)₂N(Y₁)(Y₂), wherein Y₁ and Y₂ can be the same or different and are independently selected from the group consisting of hydrogen, alkyl, aryl, cycloalkyl, and -alkylene-aryl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂- and the like which form moieties such as, for example:

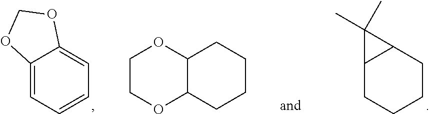

The term “silylalkyl,” as used herein, refers to an alkyl group as defined above, wherein one or more of the alkyl group's hydrogen atoms has been replaced with a —Si(RX)₃ group, wherein each occurrence of Rx is independently C₁-C₆ alkyl, phenyl or a 3 to 6-membered cycloalkyl group. In one embodiment, a silylalkyl group has from 1 to 6 carbon atoms. In another embodiment, a silyl alkyl group contains a —Si(CH₃)₃ moiety. Non-limiting examples of silylalkyl groups include —CH₂—Si(CH₃)₃ and —CH₂CH₂—Si(CH₃)₃.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any substituent or variable (e.g., R¹, m, etc.) occurs more than one time in any constituent or in Formula (I), its definition on each occurrence is independent of its definition at every other occurrence, unless otherwise indicated.

As used herein, the term “composition” is intended to encompass a product comprising the specified ingredients in the specified amounts, as well as any product which results from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to provide a Silane-Containing Heterocyclic Compound or a pharmaceutically acceptable salt or solvate of the compound. The transformation may occur by various mechanisms (e.g., by metabolic or chemical processes), such as, for example, through hydrolysis in blood.

For example, if a Silane-Containing Heterocyclic Compound or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 6 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as (3-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a Silane-Containing Heterocyclic Compound contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, —P(O)(OH)₂, —P(O)(O(C₁-C₆)alkyl)₂ or glycosyl (the radical resulting from the removal of a hydroxyl group of the hemiacetal form of a carbohydrate), and the like.

If a Silane-Containing Heterocyclic Compound incorporates an amine functional group, a prodrug can be formed by the replacement of a hydrogen atom in the amine group with a group such as, for example, R-carbonyl-, RO-carbonyl-, NRR′-carbonyl- wherein R and R′ are each independently (C₁-C₁₀)alkyl, (C₃-C₇) cycloalkyl, benzyl, a natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl; carboxy (C₁-C₆)alkyl; amino(C₁-C₄)alkyl or mono-N- or di-N,N—(C₁-C₆)alkylaminoalkyl; —C(Y⁴)Y⁵ wherein Y⁴ is H or methyl and Y⁵ is mono-N- or di-N,N—(C₁-C₆)alkylamino morpholino; piperidin-1-yl or pyrrolidin-1-yl, and the like.

Pharmaceutically acceptable esters of the present compounds include the following groups: (1) carboxylic acid esters obtained by esterification of the hydroxy group of a hydroxyl compound, in which the non-carbonyl moiety of the carboxylic acid portion of the ester grouping is selected from straight or branched chain alkyl (e.g., methyl, ethyl, n-propyl, isopropyl, t-butyl, sec-butyl or n-butyl), alkoxyalkyl (e.g., methoxymethyl), aralkyl (e.g., benzyl), aryloxyalkyl (for example, phenoxymethyl), aryl (e.g., phenyl optionally substituted with, for example, halogen, C₁₋₄alkyl, —O—(C₁₋₄alkyl) or amino); (2) sulfonate esters, such as alkyl- or aralkylsulfonyl (for example, methanesulfonyl); (3) amino acid esters (e.g., L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

The Silane-Containing Heterocyclic Compounds can form salts which are also within the scope of this invention. Reference to a Silane-Containing Heterocyclic Compound herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a Silane-Containing Heterocyclic Compound contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the Compounds of Formula (I) may be formed, for example, by reacting a Silane-Containing Heterocyclic Compound with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Diastereomeric mixtures can be separated into their individual diastereomers on the basis of their physical chemical differences by methods well-known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the Silane-Containing Heterocyclic Compounds may be atropisomers (e.g., substituted biaryls) and are considered as part of this invention. Enantiomers can also be directly separated using chiral chromatographic techniques.

It is also possible that the Silane-Containing Heterocyclic Compounds may exist in different tautomeric forms, and all such forms are embraced within the scope of the invention. For example, all keto-enol and imine-enamine forms of the compounds are included in the invention.

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. If a Silane-Containing Heterocyclic Compound incorporates a double bond or a fused ring, both the cis- and trans-forms, as well as mixtures, are embraced within the scope of the invention.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to apply equally to the salt, solvate, ester and prodrug of enantiomers, stereoisomers, rotamers, tautomers, positional isomers, racemates or prodrugs of the inventive compounds.

In the Compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched Compounds of Formula (I) can be prepared without undue experimentation by conventional techniques well known to those skilled in the art or by processes analogous to those described in the Schemes and Examples herein using appropriate isotopically-enriched reagents and/or intermediates. In one embodiment, a Compound of Formula (I) has one or more of its hydrogen atoms replaced with deuterium.

Polymorphic forms of the Silane-Containing Heterocyclic Compounds, and of the salts, solvates, hydrates, esters and prodrugs of the Silane-Containing Heterocyclic Compounds, are intended to be included in the present invention.

The following abbreviations are used below and have the following meanings: Ac is acyl; AcCl is acetyl chloride; AcOH or HOAc is acetic acid; Amphos is (4-(N,N)-dimethylaminophenyl)-di-tertbutylphosphine; Aq is aqueous; BF₃.OEt₂ is boron trifluoride etherate; BOC or Boc is tert-butyloxycarbonyl; Boc₂O is Boc anhydride; Boc-Pro-OH is Boc protected proline; L-Boc-Val-OH is Boc protected L-valine; BOP is Benzotriazole-1-yl-oxy-tris-(dimethylamino)-phosphonium hexafluorophosphate; n-BuLi is n-butyllithium; CBZ or Cbz is carbobenzoxy; DCM is dichloromethane; DDQ is 2,3-dichloro-5,6-dicyano-1,4-benzoquinone; Dess-Martin reagent is, 1,1-Triacetoxy-1,1-dihydro-1,2-benziodoxol-3(1H)-one; DIPEA is diisopropylethylamine; DME is dimethoxyethane; DMF is N,N-dimethylformamide; dppf is diphenylphosphinoferrocene; DMSO is dimethylsulfoxide; EtMgBr is ethylmagnesium bromide; EtOAc is ethyl acetate; Et₂O is diethyl ether; Et₃N or NEt₃ is triethylamine; HATU is O-(7-azabenzotriazol-1-yl)-N,N,N′,N′-tetramethyluronium hexafluorophosphate; HPLC is high performance liquid chromatography; HRMS is high resolution mass spectrometry; KOAc is potassium acetate; LCMS is liquid chromatography/mass spectrometry; LiHMDS is lithium hexamethyldisilazide; LRMS is low resolution mass spectrometry; MeI is iodomethane; MeOH is methanol; NBS is N-bromosuccinimide; NH₄OAc is ammonium acetate; NMM is N-methylmorpholine; Pd/C is palladium on carbon; Pd(PPh₃)₄ is tetrakis (triphenylphosphine)palladium(0); PdCl₂(dppf)₂ is [1,1′-Bis(diphenylphosphino) ferrocene]dichloro palladium(II); PdCl₂(dppf)₂*CH₂Cl₂ is [1,1′-Bis(diphenylphosphino)ferrocene] dichloro palladium(II) complex with dichloromethane; pinacol₂B₂ is bis(pinacolato)diboron; PPTS is pyridinium p-toluene sulfonate; RPLC is reverse-phase liquid chromatography; Select-F is 1-Chloromethyl-4-Fluoro-1, 4-Diazoniabicyclo[2.2.2]Octane Bis-(Tetrafluoroborate); SEM-Cl is 2-(trimethylsilyl)ethoxymethyl chloride; TBAF is tetrabutylammonium fluoride; TBDMSCl is tert-butyldimethylsilyl chloride; TFA is trifluoroacetic acid; Tf₂O is triflic anhydride; THF is tetrahydrofuran; TLC is thin-layer chromatography; and TosCl is p-toluenesulfonyl chloride.

### The Compounds of Formula (I)

The present invention provides Silane-Containing Heterocyclic Compounds of Formula (I):

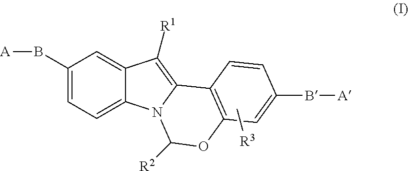

and pharmaceutically acceptable salts thereof, wherein A, A′, B, B′, R¹ R² and R³ are as defined above for the Compounds of Formula (I).

In one embodiment, R¹ is H.

In another embodiment, R¹ is halo.

In one embodiment, R² is 5 or 6-membered monocyclic heteroaryl, optionally substituted with R⁶.

In another embodiment, R² is 5 or 6-membered monocyclic heteroaryl, optionally substituted with C₃-C₇ cycloalkyl.

In another embodiment, R² is 5 or 6-membered monocyclic heteroaryl which is substituted with a cyclopropyl group.

In still another embodiment, R² is 9 or 10-membered bicyclic heteroaryl, optionally substituted with R⁶.

In another embodiment, R² is 9 or 10-membered bicyclic heteroaryl, optionally substituted with C₃-C₇ cycloalkyl.

In one embodiment, one occurrence of R⁴ is:

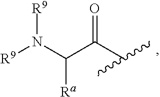

wherein R^{a} is selected from C₁-C₆ alkyl, C₃-C₆ cycloalkyl, phenyl, C₁-C₆ haloalkyl and 4 to 6-membered monocyclic heterocycloalkyl, wherein said 4 to 6-membered monocyclic heterocycloalkyl group can be optionally substituted with up to five groups, each independently selected from halo, C₁-C₆ alkyl and C₃-C₇ cycloalkyl; and R⁹ is H or C₁-C₆ alkyl.

In another embodiment, one occurrence of R⁴ is:

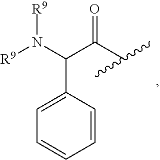

wherein each occurrence of R⁹ is H or methyl.

In one embodiment, for the compounds of formula (I) or (Ia), each occurrence of R⁴ is independently:

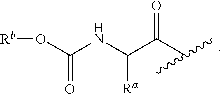

In one embodiment, B is:

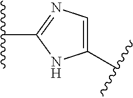 [SMILES: *c1cnc(*)[nH]1]

### and B′ is:

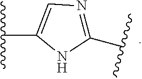 [SMILES: *c1cnc(*)[nH]1]

In one embodiment, one of A and A′ is a 5-membered heterocycloalkyl group.

In another embodiment, one of A and A′ is a 6-membered heterocycloalkyl group.

In another embodiment, one of A and A′ is selected from:

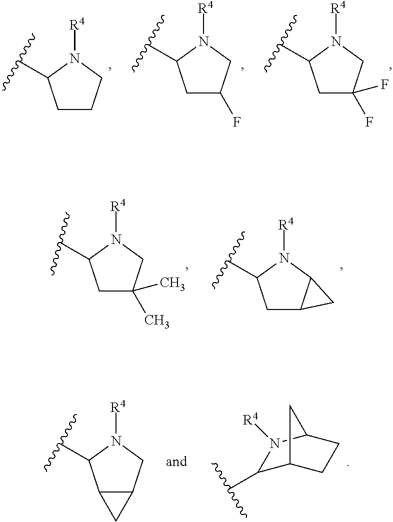

In another embodiment, one of A and A′ is selected from:

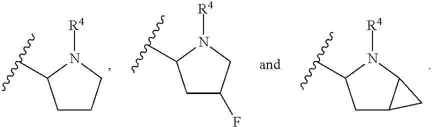

In yet another embodiment, one of A and A′ is:

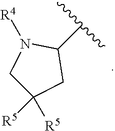

wherein each occurrence of R⁵ is independently H or F.

In another embodiment, one of A and A′ is:

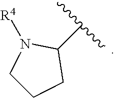

In one embodiment, one of A and A′ is R¹.

In another embodiment, each of A and A′ are independently R¹¹.

In yet another embodiment, one of A and A′ is:

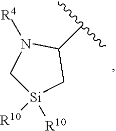

wherein each occurrence of each occurrence of R¹⁰ is independently selected from C₁-C₆ alkyl.

In yet another embodiment, one of A and A′ is:

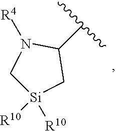

wherein each R¹⁰ group, together with the common silicon atom to which they are attached, join to form a 4- to 7-membered silyl-containing monocyclic heterocycloalkyl ring.

In another embodiment, one of A and A′ is:

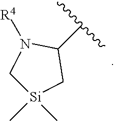

In one embodiment, A and A′ are each independently selected from:

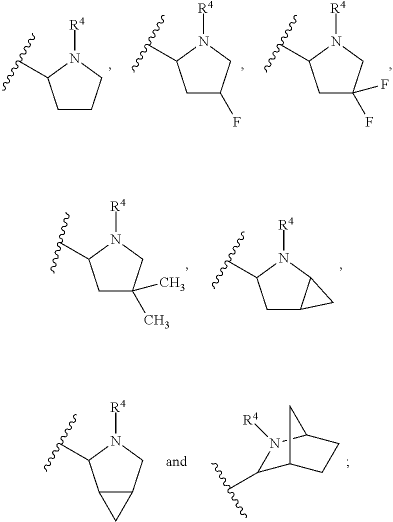

and each occurrence of R⁴ is independently:

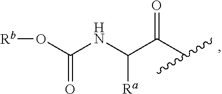

wherein R^{a} is isopropyl, phenyl, cyclopropyl,

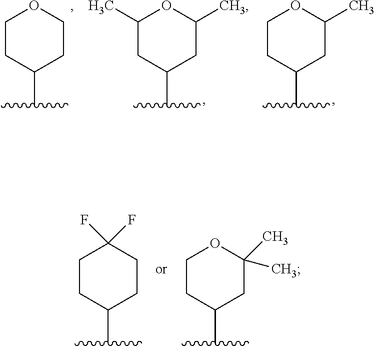

and R^{b} is C₁-C₆ alkyl.

In another embodiment, A and A′ are each independently selected from:

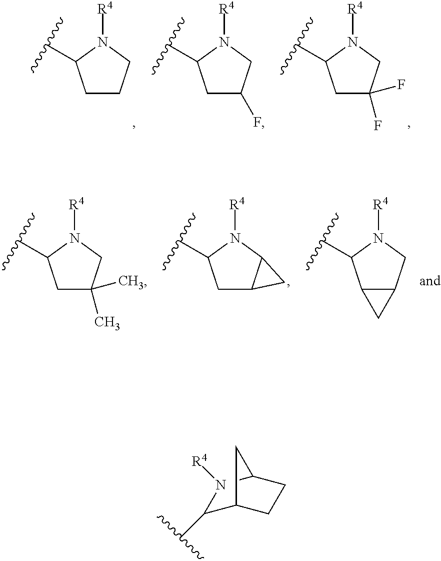

### and R⁴ is:

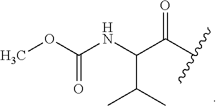 [SMILES: *C(=O)C(NC(=O)OC)C(C)C]

In another embodiment, A and A′ are each independently selected from:

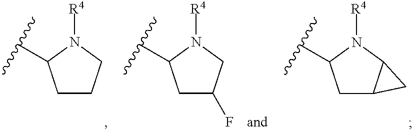

each occurrence of R⁴ is independently:

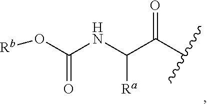

wherein R^{a} is isopropyl, phenyl, cyclopropyl,

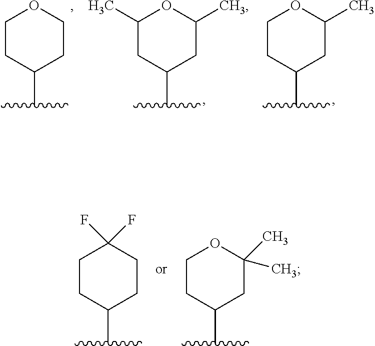

and R^{b} is methyl.

In one embodiment, one of A and A′ is selected from:

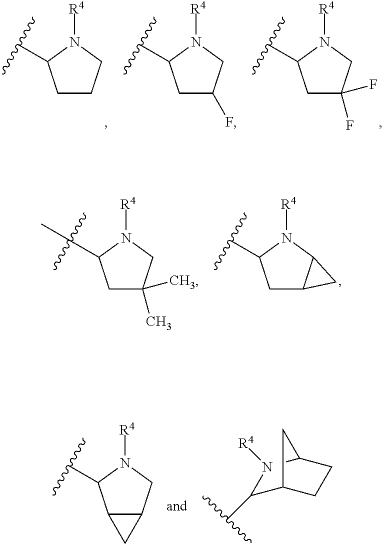

and the other of A and A′ is selected from:

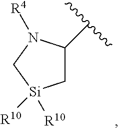

wherein each occurrence of R¹⁰ is independently selected from C₁-C₆ alkyl.

In another embodiment, one of A and A′ is selected from:

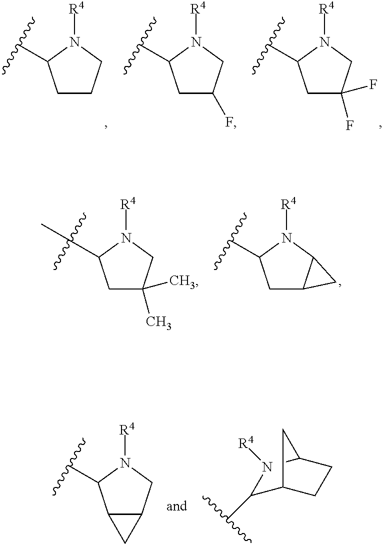

and the other of A and A′ is selected from:

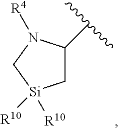

wherein each R¹⁰ group, together with the common silicon atom to which they are attached, join to form a 4- to 7-membered silyl-containing monocyclic heterocycloalkyl ring.

In one embodiment, one of A and A′ is selected from:

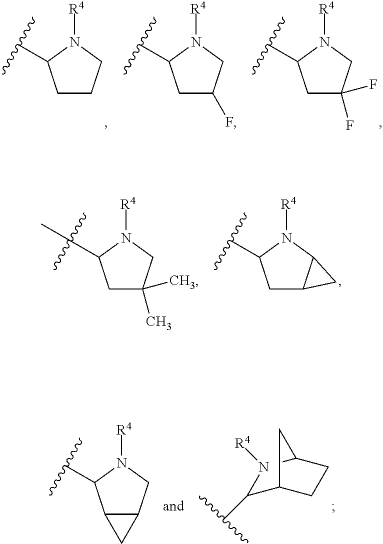

the other of A and A′ is selected from:

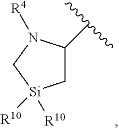

wherein each occurrence of R¹⁰ is independently selected from C₁-C₆ alkyl; and each occurrence of R⁴ is independently:

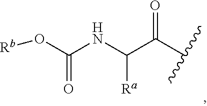

wherein R^{a} is selected from C₁-C₆ alkyl, C₃-C₆ cycloalkyl, phenyl, C₁-C₆ haloalkyl and 4 to 6-membered monocyclic heterocycloalkyl, wherein said 4 to 6-membered monocyclic heterocycloalkyl group can be optionally substituted with up to five groups, each independently selected from halo, C₁-C₆ alkyl and C₃-C₇ cycloalkyl; and R^{b} is C₁-C₆ alkyl.

In another embodiment, one of A and A′ is selected from:

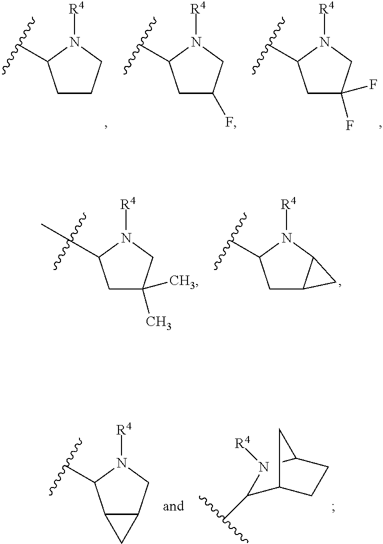

the other of A and A′ is selected from:

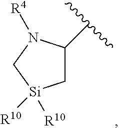

wherein each R¹⁰ group, together with the common silicon atom to which they are attached, join to form a 4- to 7-membered silyl-containing monocyclic heterocycloalkyl ring; and each occurrence of R⁴ is independently:

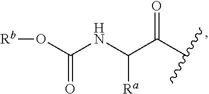

wherein R^{a} is selected from C₁-C₆ alkyl, C₃-C₆ cycloalkyl, phenyl, C₁-C₆ haloalkyl and 4 to 6-membered monocyclic heterocycloalkyl, wherein said 4 to 6-membered monocyclic heterocycloalkyl group can be optionally substituted with up to five groups, each independently selected from halo, C₁-C₆ alkyl and C₃-C₇ cycloalkyl; and R^{b} is C₁-C₆ alkyl.

In one embodiment, variables A, A′, B, B′, R¹, R² and R³ for the Compounds of Formula (I) are selected independently of each other.

In another embodiment, the Compounds of Formula (I) are in substantially purified form.

In one embodiment, the Compounds of Formula (I) have the formula (Ia):

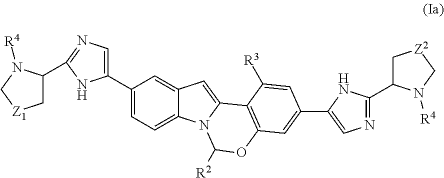

wherein:

R² is 5-membered heteroaryl, which can be optionally substituted with C₁-C₆ alkyl or C₃-C₇ cycloalkyl;

R³ is H or halo;

each occurrence of R⁴ is:

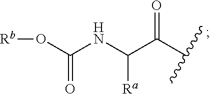

each occurrence of R^{a} is independently selected from C₁-C₆ alkyl, C₃-C₆ cycloalkyl, 4- to 7-membered monocyclic heterocycloalkyl and phenyl, wherein said 4- to 7-membered monocyclic heterocycloalkyl group can be optionally substituted with up to 4 substituents, each of which are independently selected from halo and C₁-C₆ alkyl;

each occurrence of R^{b} is independently selected from C₁-C₆ alkyl;

Z¹ is —Si(R¹⁰)₂— or —C(R⁵)₂—;

Z² is —Si(R¹⁰)₂— or —C(R⁵)₂-;

each occurrence of R⁵ is independently H or F, or or two R⁵ groups that are attached to the same carbon atom, combine to form a spirocyclic C₃-C₅ cycloalkyl group;

each occurrence of R¹⁰ is independently C₁-C₆ alkyl, or two R¹⁰ groups that are attached to the same Si atom, combine to form a —(CH₂)₄- or —(CH₂)₅— group; and

such that at least one of Z¹ and Z² is Si(R¹⁰)₂.

In one embodiment, for the compounds of formula (Ia), Z¹ is Si(R¹⁰)₂ and Z² is CH₂, CH(F) or CF₂.

In another embodiment, for the compounds of formula (Ia), Z² is Si(R¹⁰)₂ and Z¹ is CH₂, CH(F) or CF₂.

In another embodiment, for the compounds of formula (Ia), Z¹ and Z² are each independently Si(R¹⁰)₂.

In a further embodiment, for the compounds of formula (I) or (Ia), R² is thiadiazolyl, which is substituted with a cyclopropyl group.

In one embodiment, for the compounds of formula (I) or (Ia): R² is:

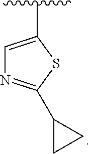 [SMILES: *c1cnc(C2CC2)s1]

In one embodiment, for the compounds of formula (I) or (Ia), R³ is halo.

In another embodiment, for the compounds of formula (I) or (Ia), R³ is F.

In one embodiment, for the compounds of formula (I) or (Ia), each occurrence of R⁴ is independently:

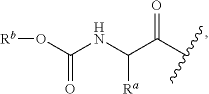

wherein R^{a} is selected from C₁-C₆ alkyl, C₃-C₆ cycloalkyl, phenyl, C₁-C₆haloalkyl and 4 to 6-membered monocyclic heterocycloalkyl, wherein said 4 to 6-membered monocyclic heterocycloalkyl group can be optionally substituted with up to five groups, each independently selected from halo, C₁-C₆ alkyl and C₃-C₇ cycloalkyl; and R^{b} is C₁-C₆ alkyl.

In one embodiment, for the compounds of formula (I) or (Ia), each occurrence of R⁴ is independently:

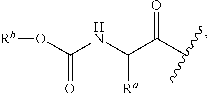

wherein R^{a} is isopropyl, phenyl, cyclopropyl,

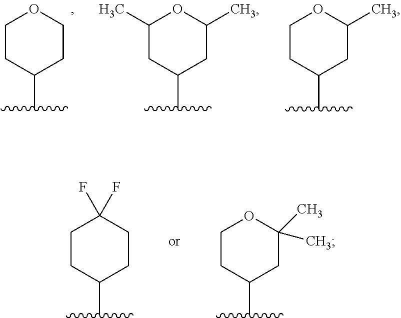

and R^{b} is C₁-C₆ alkyl.

In one embodiment, for the compounds of formula (I) or (Ia), each occurrence of R⁴ is independently:

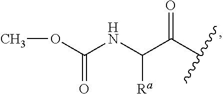

wherein R^{a} is isopropyl, phenyl, cyclopropyl,

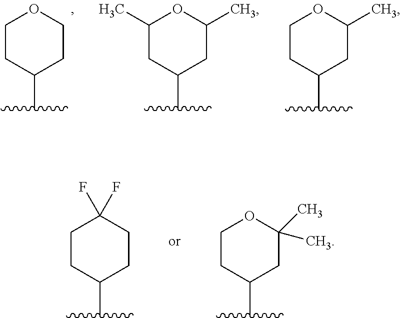

In another embodiment, for the compounds of formula (I) or (Ia), each occurrence of R⁴ is independently selected from:

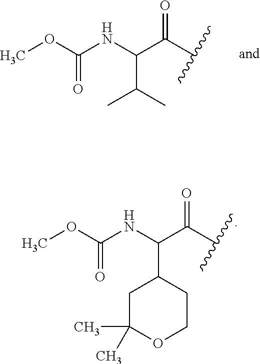 [SMILES: *C(=O)C(NC(=O)OC)C(C)C ; *C(=O)C(NC(=O)OC)C1CCOC(C)(C)C1]

In one embodiment, variables Z¹, Z², R², R³ and R⁴ for the Compounds of Formula (Ia) are selected independently of each other.

In another embodiment, the Compounds of Formula (Ia) are in substantially purified form.

Other embodiments of the present invention include the following:

(a) A pharmaceutical composition comprising an effective amount of a Compound of Formula (I) or a pharmaceutically acceptable salt thereof, and a pharmaceutically acceptable carrier.

(b) The pharmaceutical composition of (a), further comprising a second therapeutic agent selected from the group consisting of HCV antiviral agents, immunomodulators, and anti-infective agents.

(c) The pharmaceutical composition of (b), wherein the HCV antiviral agent is an antiviral selected from the group consisting of HCV protease inhibitors and HCV NS5B polymerase inhibitors.

(d) A pharmaceutical combination that is (i) a Compound of Formula (I) and (ii) a second therapeutic agent selected from the group consisting of HCV antiviral agents, immunomodulators, and anti-infective agents; wherein the Compound of Formula (I) and the second therapeutic agent are each employed in an amount that renders the combination effective for inhibiting HCV replication, or for treating HCV infection and/or reducing the likelihood or severity of symptoms of HCV infection.

(e) The combination of (d), wherein the HCV antiviral agent is an antiviral selected from the group consisting of HCV protease inhibitors and HCV NS5B polymerase inhibitors.

(f) A method of inhibiting HCV replication in a subject in need thereof which comprises administering to the subject an effective amount of a Compound of Formula (I).

(g) A method of treating HCV infection and/or reducing the likelihood or severity of symptoms of HCV infection in a subject in need thereof which comprises administering to the subject an effective amount of a Compound of Formula (I).

(h) The method of (g), wherein the Compound of Formula (I) is administered in combination with an effective amount of at least one second therapeutic agent selected from the group consisting of HCV antiviral agents, immunomodulators, and anti-infective agents.

(i) The method of (h), wherein the HCV antiviral agent is an antiviral selected from the group consisting of HCV protease inhibitors and HCV NS5B polymerase inhibitors.

(j) A method of inhibiting HCV replication in a subject in need thereof which comprises administering to the subject the pharmaceutical composition of (a), (b) or (c) or the combination of (d) or (e).

(k) A method of treating HCV infection and/or reducing the likelihood or severity of symptoms of HCV infection in a subject in need thereof which comprises administering to the subject the pharmaceutical composition of (a), (b) or (c) or the combination of (d) or (e).

The present invention also includes a compound of the present invention for use (i) in, (ii) as a medicament for, or (iii) in the preparation of a medicament for: (a) medicine; (b) inhibiting HCV replication or (c) treating HCV infection and/or reducing the likelihood or severity of symptoms of HCV infection. In these uses, the compounds of the present invention can optionally be employed in combination with one or more second therapeutic agents selected from HCV antiviral agents, anti-infective agents, and immunomodulators.

Additional embodiments of the invention include the pharmaceutical compositions, combinations and methods set forth in (a)-(k) above and the uses set forth in the preceding paragraph, wherein the compound of the present invention employed therein is a compound of one of the embodiments, aspects, classes, sub-classes, or features of the compounds described above. In all of these embodiments, the compound may optionally be used in the form of a pharmaceutically acceptable salt or hydrate as appropriate.

It is further to be understood that the embodiments of compositions and methods provided as (a) through (k) above are understood to include all embodiments of the compounds, including such embodiments as result from combinations of embodiments.

Non-limiting examples of the Compounds of Formula (I) include compounds 1-19, as set forth in the Examples below, and pharmaceutically acceptable salts thereof.

### Uses of the Silane-Containing Heterocyclic Compounds

The Silane-Containing Heterocyclic Compounds may be useful in human and veterinary medicine for treating or preventing a viral infection in a patient. In one embodiment, the Silane-Containing Heterocyclic Compounds can be inhibitors of viral replication. In another embodiment, the Silane-Containing Heterocyclic Compounds can be inhibitors of HCV replication. Accordingly, the Silane-Containing Heterocyclic Compounds may be useful for treating viral infections, such as HCV. In accordance with the invention, the Silane-Containing Heterocyclic Compounds can be administered to a patient in need of treatment or prevention of a viral infection.

Accordingly, in one embodiment, the invention provides methods for treating a viral infection in a patient comprising administering to the patient an effective amount of at least one Silane-Containing Heterocyclic Compound or a pharmaceutically acceptable salt thereof.

### Treatment or Prevention of a Flaviviridae Virus

The Silane-Containing Heterocyclic Compounds can be useful for treating or preventing a viral infection caused by the Flaviviridae family of viruses.

Examples of Flaviviridae infections that can be treated or prevented using the present methods include but are not limited to, dengue fever, Japanese encephalitis, Kyasanur Forest disease, Murray Valley encephalitis, St. Louis encephalitis, Tick-bome encephalitis, West Nile encephalitis, yellow fever and Hepatitis C Virus (HCV) infection.

In one embodiment, the Flaviviridae infection being treated is hepatitis C virus infection.

### Treatment or Prevention of HCV Infection

The Silane-Containing Heterocyclic Compounds may be useful in the inhibition of HCV replication, the treatment of HCV infection and/or reduction of the likelihood or severity of symptoms of HCV infection and the inhibition of HCV viral replication and/or HCV viral production in a cell-based system. For example, the Silane-Containing Heterocyclic Compounds may be useful in treating infection by HCV after suspected past exposure to HCV by such means as blood transfusion, exchange of body fluids, bites, accidental needle stick, or exposure to patient blood during surgery or other medical procedures.

In one embodiment, the hepatitis C infection is acute hepatitis C. In another embodiment, the hepatitis C infection is chronic hepatitis C.

Accordingly, in one embodiment, the invention provides methods for treating HCV infection in a patient, the methods comprising administering to the patient an effective amount of at least one Silane-Containing Heterocyclic Compound or a pharmaceutically acceptable salt thereof. In a specific embodiment, the amount administered is effective to treat or prevent infection by HCV in the patient. In another specific embodiment, the amount administered is effective to inhibit HCV viral replication and/or viral production in the patient.

The Silane-Containing Heterocyclic Compounds may also be useful in the preparation and execution of screening assays for antiviral compounds. For example the Silane-Containing Heterocyclic Compounds may be useful for identifying resistant HCV replicon cell lines harboring mutations within NS5A, which are excellent screening tools for more powerful antiviral compounds. Furthermore, the Silane-Containing Heterocyclic Compounds may be useful in establishing or determining the binding site of other antivirals to the HCV replicase.

The compositions and combinations of the present invention can be useful for treating a patient suffering from infection related to any HCV genotype. HCV types and subtypes may differ in their antigenicity, level of viremia, severity of disease produced, and response to interferon therapy as described in Holland et al., Pathology, 30(2):192-195 (1998). The nomenclature set forth in Simmonds et al., J Gen Virol, 74(Pt11):2391-2399 (1993) is widely used and classifies isolates into six major genotypes, 1 through 6, with two or more related subtypes, e.g., 1a and 1b. Additional genotypes 7-10 and 11 have been proposed, however the phylogenetic basis on which this classification is based has been questioned, and thus types 7, 8, 9 and 11 isolates have been reassigned as type 6, and type 10 isolates as type 3 (see Lamballerie et al., J Gen Virol, 78(Pt1):45-51 (1997)). The major genotypes have been defined as having sequence similarities of between 55 and 72% (mean 64.5%), and subtypes within types as having 75%-86% similarity (mean 80%) when sequenced in the NS-5 region (see Simmonds et al., J Gen Virol, 75(Pt 5):1053-1061 (1994)).

### Combination Therapy

In another embodiment, the present methods for treating or preventing HCV infection can further comprise the administration of one or more additional therapeutic agents which are not Silane-Containing Heterocyclic Compounds.

In one embodiment, the additional therapeutic agent is an antiviral agent.

In another embodiment, the additional therapeutic agent is an immunomodulatory agent, such as an immunosuppressive agent.

Accordingly, in one embodiment, the present invention provides methods for treating a viral infection in a patient, the method comprising administering to the patient: (i) at least one Silane-Containing Heterocyclic Compound, or a pharmaceutically acceptable salt thereof, and (ii) at least one additional therapeutic agent that is other than a Silane-Containing Heterocyclic Compound, wherein the amounts administered are together effective to treat or prevent a viral infection.

When administering a combination therapy of the invention to a patient, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, a Silane-Containing Heterocyclic Compound and an additional therapeutic agent may be present in fixed amounts (dosage amounts) in a single dosage unit (e.g., a capsule, a tablet and the like).

In one embodiment, the at least one Silane-Containing Heterocyclic Compound is administered during a time when the additional therapeutic agent(s) exert their prophylactic or therapeutic effect, or vice versa.

In another embodiment, the at least one Silane-Containing Heterocyclic Compound and the additional therapeutic agent(s) are administered in doses commonly employed when such agents are used as monotherapy for treating a viral infection.

In another embodiment, the at least one Silane-Containing Heterocyclic Compound and the additional therapeutic agent(s) are administered in doses lower than the doses commonly employed when such agents are used as monotherapy for treating a viral infection.

In still another embodiment, the at least one Silane-Containing Heterocyclic Compound and the additional therapeutic agent(s) act synergistically and are administered in doses lower than the doses commonly employed when such agents are used as monotherapy for treating a viral infection.

In one embodiment, the at least one Silane-Containing Heterocyclic Compound and the additional therapeutic agent(s) are present in the same composition. In one embodiment, this composition is suitable for oral administration. In another embodiment, this composition is suitable for intravenous administration. In another embodiment, this composition is suitable for subcutaneous administration. In still another embodiment, this composition is suitable for parenteral administration.

Viral infections and virus-related disorders that can be treated or prevented using the combination therapy methods of the present invention include, but are not limited to, those listed above.

In one embodiment, the viral infection is HCV infection.

The at least one Silane-Containing Heterocyclic Compound and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy.

In one embodiment, the administration of at least one Silane-Containing Heterocyclic Compound and the additional therapeutic agent(s) may inhibit the resistance of a viral infection to these agents.

Non-limiting examples of additional therapeutic agents useful in the present compositions and methods include an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, an antibody therapy (monoclonal or polyclonal), and any agent useful for treating an RNA-dependent polymerase-related disorder.

In one embodiment, the additional therapeutic agent is a viral protease inhibitor.

In another embodiment, the additional therapeutic agent is a viral replication inhibitor.

In another embodiment, the additional therapeutic agent is an HCV NS3 protease inhibitor.

In still another embodiment, the additional therapeutic agent is an HCV NS5B polymerase inhibitor.

In another embodiment, the additional therapeutic agent is a nucleoside inhibitor.

In another embodiment, the additional therapeutic agent is an interferon.

In yet another embodiment, the additional therapeutic agent is an HCV replicase inhibitor.

In another embodiment, the additional therapeutic agent is an antisense agent.

In another embodiment, the additional therapeutic agent is a therapeutic vaccine.

In a further embodiment, the additional therapeutic agent is a virion production inhibitor.

In another embodiment, the additional therapeutic agent is an antibody therapy.

In another embodiment, the additional therapeutic agent is an HCV NS2 inhibitor.

In still another embodiment, the additional therapeutic agent is an HCV NS4A inhibitor.

In another embodiment, the additional therapeutic agent is an HCV NS4B inhibitor.

In another embodiment, the additional therapeutic agent is an HCV NS5A inhibitor

In yet another embodiment, the additional therapeutic agent is an HCV NS3 helicase inhibitor.

In another embodiment, the additional therapeutic agent is an HCV IRES inhibitor.

In another embodiment, the additional therapeutic agent is an HCV p7 inhibitor.

In a further embodiment, the additional therapeutic agent is an HCV entry inhibitor.

In another embodiment, the additional therapeutic agent is an HCV assembly inhibitor.

In one embodiment, the additional therapeutic agents comprise a viral protease inhibitor and a viral polymerase inhibitor.

In still another embodiment, the additional therapeutic agents comprise a viral protease inhibitor and an immunomodulatory agent.

In yet another embodiment, the additional therapeutic agents comprise a polymerase inhibitor and an immunomodulatory agent.

In another embodiment, the additional therapeutic agents comprise a viral protease inhibitor and a nucleoside.

In another embodiment, the additional therapeutic agents comprise an immunomodulatory agent and a nucleoside.

In one embodiment, the additional therapeutic agents comprise an HCV protease inhibitor and an HCV polymerase inhibitor.

In another embodiment, the additional therapeutic agents comprise a nucleoside and an HCV NS5A inhibitor.

In another embodiment, the additional therapeutic agents comprise a viral protease inhibitor, an immunomodulatory agent and a nucleoside.

In a further embodiment, the additional therapeutic agents comprise a viral protease inhibitor, a viral polymerase inhibitor and an immunomodulatory agent.

In another embodiment, the additional therapeutic agent is ribavirin.

HCV polymerase inhibitors useful in the present compositions and methods include, but are not limited to, VP-19744 (Wyeth/ViroPharma), PSI-7851 (Pharmasset), MK-3682 (Merck), GS-7977 (sofosbuvir, Gilead), R7128 (Roche/Pharmasset), PF-868554/filibuvir (Pfizer), VCH-759 (ViroChem Pharma), HCV-796 (Wyeth/ViroPharma), IDX-184 (Idenix), IDX-375 (Idenix), NM-283 (Idenix/Novartis), R-1626 (Roche), MK-0608 (Isis/Merck), 1NX-8014 (Inhibitex), INX-8018 (Inhibitex), INX-189 (Inhibitex), GS 9190 (Gilead), A-848837 (Abbott), ABT-333 (Abbott), ABT-072 (Abbott), A-837093 (Abbott), BI-207127 (Boehringer-Ingelheim), BILB-1941 (Boehringer-Ingelheim), MK-3281 (Merck), VCH222 (ViroChem), VCH916 (ViroChem), VCH716(ViroChem), GSK-71185 (Glaxo SmithKline), ANA598 (Anadys), GSK-625433 (Glaxo SmithKline), XTL-2125 (XTL Biopharmaceuticals), and those disclosed in Ni et al., Current Opinion in Drug Discovery and Development, 7(4):446 (2004); Tan et al., Nature Reviews, 1:867 (2002); and Beaulieu et al., Current Opinion in Investigational Drugs, 5:838 (2004).

Other HCV polymerase inhibitors useful in the present compositions and methods include, but are not limited to, those disclosed in PCT International Publication Nos. WO 08/082484, WO 08/082488, WO 08/083351, WO 08/136815, WO 09/032116, WO 09/032123, WO 09/032124 and WO 09/032125.

Interferons useful in the present compositions and methods include, but are not limited to, interferon alfa-2a, interferon alfa-2b, interferon alfacon-1 and PEG-interferon alpha conjugates. “PEG-interferon alpha conjugates” are interferon alpha molecules covalently attached to a PEG molecule. Illustrative PEG-interferon alpha conjugates include interferon alpha-2a (Roferon™, Hoffman La-Roche, Nutley, N.J.) in the form of pegylated interferon alpha-2a (e.g., as sold under the trade name Pegasys™), interferon alpha-2b (Intron™, from Schering-Plough Corporation) in the form of pegylated interferon alpha-2b (e.g., as sold under the trade name PEG-Intron™ from Schering-Plough Corporation), interferon alpha-2b-XL (e.g., as sold under the trade name PEG-Intron™), interferon alpha-2c (Berofor Alpha™ Boehringer Ingelheim, Ingelheim, Germany), PEG-interferon lambda (Bristol-Myers Squibb and ZymoGenetics), interferon alfa-2b alpha fusion polypeptides, interferon fused with the human blood protein albumin (Albuferon™, Human Genome Sciences), Omega Interferon (Intarcia), Locteron controlled release interferon (Biolex/OctoPlus), Biomed-510 (omega interferon), Peg-IL-29 (ZymoGenetics), Locteron CR (Octoplus), IFN-α-2b-XL (Flamel Technologies), and consensus interferon as defined by determination of a consensus sequence of naturally occurring interferon alphas (Infergen™, Amgen, Thousand Oaks, Calif.).

Antibody therapy agents useful in the present compositions and methods include, but are not limited to, antibodies specific to IL-10 (such as those disclosed in US Patent Publication No. US2005/0101770, humanized 12G8, a humanized monoclonal antibody against human IL-10, plasmids containing the nucleic acids encoding the humanized 12G8 light and heavy chains were deposited with the American Type Culture Collection (ATCC) as deposit numbers PTA-5923 and PTA-5922, respectively), and the like).

Examples of viral protease inhibitors useful in the present compositions and methods include, but are not limited to, an HCV protease inhibitor.

HCV protease inhibitors useful in the present compositions and methods include, but are not limited to, those disclosed in U.S. Pat. Nos. 7,494,988, 7,485,625, 7,449,447, 7,442,695, 7,425,576, 7,342,041, 7,253,160, 7,244,721, 7,205,330, 7,192,957, 7,186,747, 7,173,057, 7,169,760, 7,012,066, 6,914,122, 6,911,428, 6,894,072, 6,846,802, 6,838,475, 6,800,434, 6,767,991, 5,017,380, 4,933,443, 4,812,561 and 4,634,697; U.S. Patent Publication Nos. US20020068702, US20020160962, US20050119168, US20050176648, US20050209164, US20050249702 and US20070042968; and PCT International Publication Nos. WO 03/006490, WO 03/087092, WO 04/092161 and WO 08/124148.

Additional HCV protease inhibitors useful in the present compositions and methods include, but are not limited to, SCH503034 (Boceprevir, Schering-Plough), SCH900518 (Schering-Plough), MK-5172 (Merck), VX-950 (Telaprevir, Vertex), VX-500 (Vertex), VX-813 (Vertex), VBY-376 (Virobay), BI-201335 (Boehringer Ingelheim), TMC-435 (Medivir/Tibotec), ABT-450 (Abbott), TMC-435350 (Medivir), ITMN-191/R7227 (InterMune/Roche), EA-058 (Abbott/Enanta), EA-063 (Abbott/Enanta), GS-9132 (Gilead/Achillion), ACH-1095 (Gilead/Achillon), IDX-136 (Idenix), IDX-316 (Idenix), ITMN-8356 (InterMune), ITMN-8347 (InterMune), ITMN-8096 (InterMune), ITMN-7587 (InterMune), BMS-650032 (Bristol-Myers Squibb), VX-985 (Vertex) and PHX1766 (Phenomix).

Further examples of HCV protease inhbitors useful in the present compositions and methods include, but are not limited to, those disclosed in Landro et al., Biochemistry, 36(31):9340-9348 (1997); Ingallinella et al., Biochemistry, 37(25):8906-8914 (1998); Llinas-Brunet et al., Bioorg Med Chem Lett, 8(13): 1713-1718 (1998); Martin et al., Biochemistry, 37(33):11459-11468 (1998); Dimasi et al., J Virol, 71(10):7461-7469 (1997); Martin et al., Protein Eng, 10(5):607-614 (1997); Elzouki et al., J Hepat, 27(1):42-48 (1997); Bio World Today, 9(217):4 (Nov. 10, 1998); U.S. Patent Publication Nos. US2005/0249702 and US 2007/0274951; and PCT International Publication Nos. WO 98/14181, WO 98/17679, WO 98/17679, WO 98/22496 and WO 99/07734 and WO 05/087731.

Further examples of HCV protease inhibitors useful in the present compositions and methods include, but are not limited to, MK-5172 (Merck) and the following compounds:

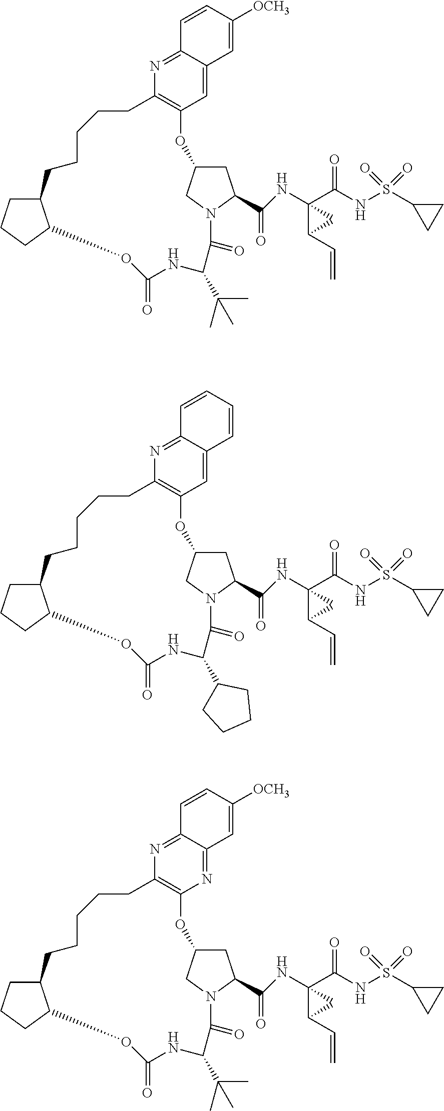

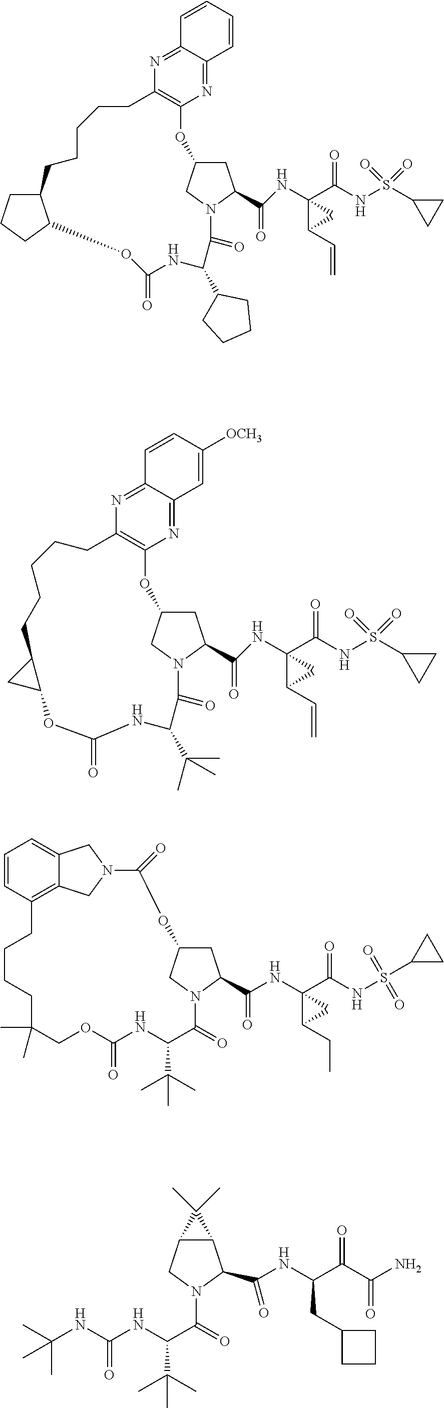

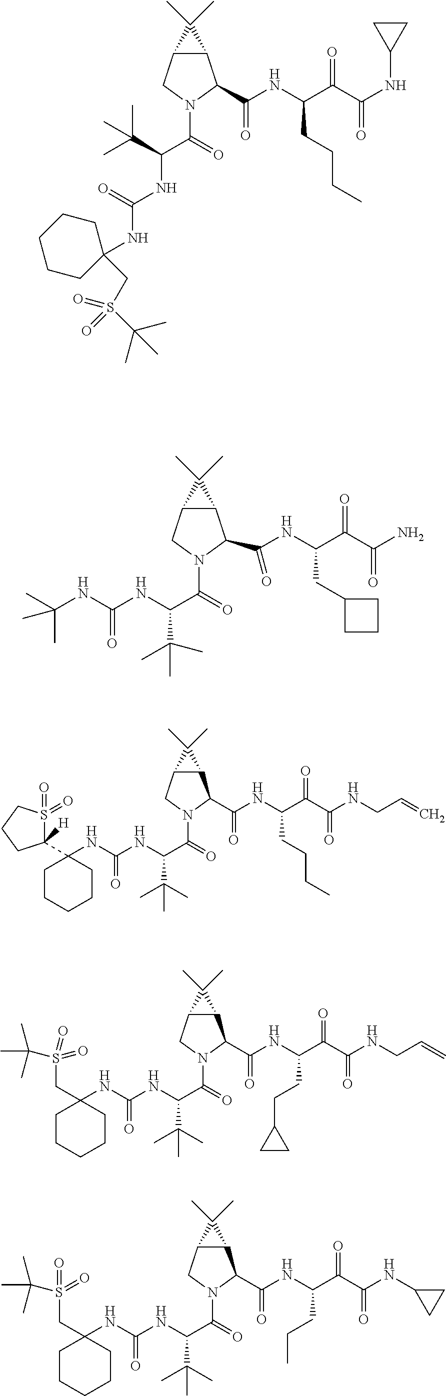

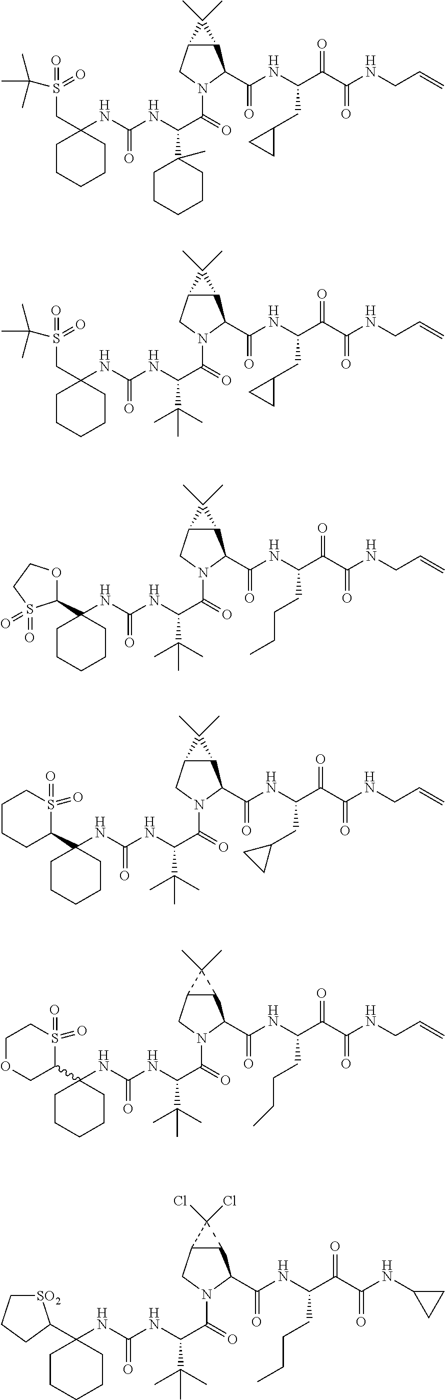

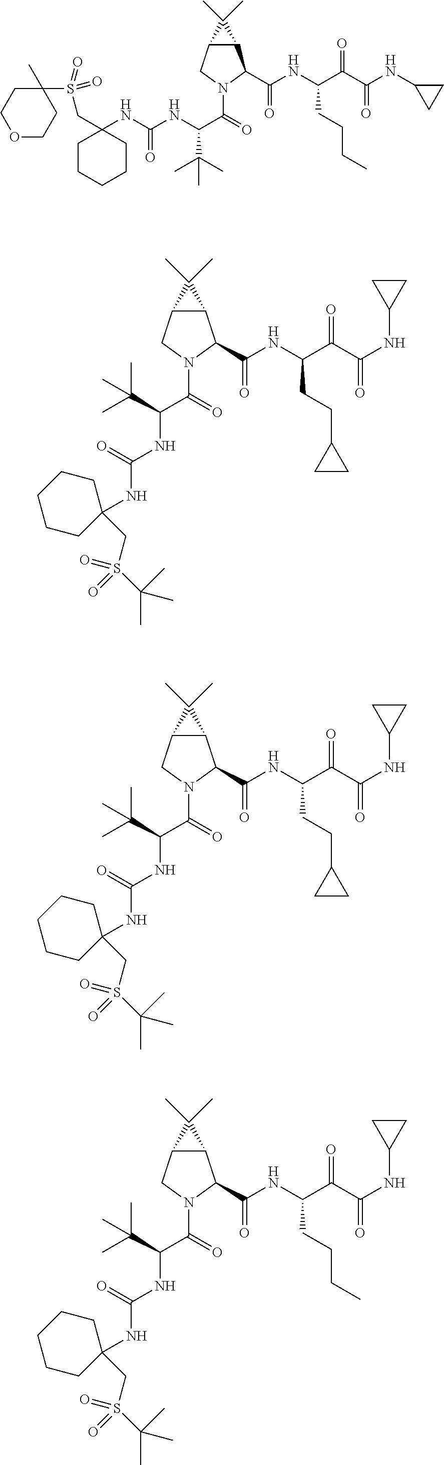 [SMILES: CCCC[C@H](NC(=O)[C@@H]1[C@@H]2[C@H](CN1C(=O)[C@@H](NC(=O)NC1(CS(=O)(=O)C3(C)CCOCC3)CCCCC1)C(C)(C)C)C2(C)C)C(=O)C(=O)NC1CC1 ; CC(C)(C)[C@H](NC(=O)NC1(CS(=O)(=O)C(C)(C)C)CCCCC1)C(=O)N1C[C@H]2[C@@H]([C@H]1C(=O)N[C@H](CCC1CC1)C(=O)C(=O)NC1CC1)C2(C)C ; CC(C)(C)[C@H](NC(=O)NC1(CS(=O)(=O)C(C)(C)C)CCCCC1)C(=O)N1C[C@H]2[C@@H]([C@H]1C(=O)N[C@@H](CCC1CC1)C(=O)C(=O)NC1CC1)C2(C)C ; CCCC[C@H](NC(=O)[C@@H]1[C@@H]2[C@H](CN1C(=O)[C@@H](NC(=O)NC1(CS(=O)(=O)C(C)(C)C)CCCCC1)C(C)(C)C)C2(C)C)C(=O)C(=O)NC1CC1]

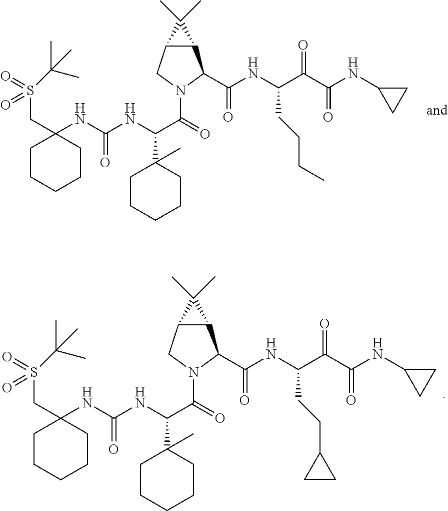 [SMILES: CCCC[C@H](NC(=O)[C@@H]1[C@@H]2[C@H](CN1C(=O)[C@@H](NC(=O)NC1(CS(=O)(=O)C(C)(C)C)CCCCC1)C1(C)CCCCC1)C2(C)C)C(=O)C(=O)NC1CC1 ; CC1([C@H](NC(=O)NC2(CS(=O)(=O)C(C)(C)C)CCCCC2)C(=O)N2C[C@H]3[C@@H]([C@H]2C(=O)N[C@@H](CCC2CC2)C(=O)C(=O)NC2CC2)C3(C)C)CCCCC1]

HCV viral replication inhibitors useful in the present compositions and methods include, but are not limited to, HCV replicase inhibitors, IRES inhibitors, NS4A inhibitors, NS3 helicase inhibitors, NS3 protease inhibitors, NS5A inhibitors, NS5B inhibitors, ribavirin, AZD-2836 (Astra Zeneca), BMS-790052 (Bristol-Myers Squibb, see Gao et al., Nature, 465:96-100 (2010)), viramidine, A-831 (Arrow Therapeutics); an antisense agent or a therapeutic vaccine.

HCV NS4A inhibitors useful in the present compositions and methods include, but are not limited to, those disclosed in U.S. Pat. Nos. 7,476,686 and 7,273,885; U.S. Patent Publication No. US20090022688; and PCT International Publication Nos. WO 2006/019831 and WO 2006/019832. Additional HCV NS4A inhibitors useful in the present compositions and methods include, but are not limited to, AZD2836 (Astra Zeneca) and ACH-806 (Achillon Pharmaceuticals, New Haven, Conn.).

HCV replicase inhibitors useful in the present compositions and methods include, but are not limited to, those disclosed in U.S. Patent Publication No. US20090081636.

Therapeutic vaccines useful in the present compositions and methods include, but are not limited to, IC41 (Intercell Novartis), CSL123 (Chiron/CSL), GI 5005 (Globeimmune), TG-4040 (Transgene), GNI-103 (GENimmune), Hepavaxx C (ViRex Medical), ChronVac-C(Inovio/Tripep), PeviPRO™ (Pevion Biotect), HCV/MF59 (Chiron/Novartis) and Civacir (NABI).

Examples of further additional therapeutic agents useful in the present compositions and methods include, but are not limited to, Ritonavir (Abbott), TT033 (Benitec/Tacere Bio/Pfizer), Sima-034 (Sima Therapeutics), GNI-104 (GENimmune), GI-5005 (GlobeImmune), IDX-102 (Idenix), Levovirin™ (ICN Pharmaceuticals, Costa Mesa, Calif.); Humax (Genmab), ITX-2155 (Ithrex/Novartis), PRO 206 (Progenics), HepaCide-I (NanoVirocides), MX3235 (Migenix), SCY-635 (Scynexis); KPE02003002 (Kemin Pharma), Lenocta (VioQuest Pharmaceuticals), IET—Interferon Enhancing Therapy (Transition Therapeutics), Zadaxin (SciClone Pharma), VP 50406™ (Viropharma, Incorporated, Exton, Pa.); Taribavirin (Valeant Pharmaceuticals); Nitazoxanide (Romark); Debio 025 (Debiopharm); GS-9450 (Gilead); PF-4878691 (Pfizer); ANA773 (Anadys); SCV-07 (SciClone Pharmaceuticals); NIM-881 (Novartis); ISIS 14803™ (ISIS Pharmaceuticals, Carlsbad, Calif.); Heptazyme™ (Ribozyme Pharmaceuticals, Boulder, Colo.); Thymosin™ (SciClone Pharmaceuticals, San Mateo, Calif.); Maxamine™ (Maxim Pharmaceuticals, San Diego, Calif.); NKB-122 (JenKen Bioscience Inc., North Carolina); Alinia (Romark Laboratories), INFORM-1 (a combination of R7128 and ITMN-191); and mycophenolate mofetil (Hoffman-LaRoche, Nutley, N.J.).

The doses and dosage regimen of the other agents used in the combination therapies of the present invention for the treatment or prevention of HCV infection can be determined by the attending clinician, taking into consideration the approved doses and dosage regimen in the package insert; the age, sex and general health of the patient; and the type and severity of the viral infection or related disease or disorder. When administered in combination, the Silane-Containing Heterocyclic Compound(s) and the other agent(s) can be administered simultaneously (i.e., in the same composition or in separate compositions one right after the other) or sequentially. This is *particularly useful when the components of the combination are given on different dosing schedules, e.g., one component is administered once daily and another component is administered every six hours, or when the preferred pharmaceutical compositions are different, e.g., one is a tablet and one is a capsule. A kit comprising the separate dosage forms is therefore advantageous.

In a further embodiment, when the additional therapeutic agent is Ribavirin (commercially available as REBETOL ribavirin from Schering-Plough or COPEGUS ribavirin from Hoffmann-La Roche), this agent is administered at a daily dosage of from about 600 to about 1400 mg/day for at least 24 weeks.

In one embodiment, one or more compounds of the present invention are administered with one or more additional therapeutic agents selected from: an immunomodulator, a viral replication inhibitor, an antisense agent, a therapeutic vaccine, a viral polymerase inhibitor, a nucleoside inhibitor, a viral protease inhibitor, a viral helicase inhibitor, a viral polymerase inhibitor a virion production inhibitor, a viral entry inhibitor, a viral assembly inhibitor, an antibody therapy (monoclonal or polyclonal), and any agent useful for treating an RNA-dependent polymerase-related disorder.

In another embodiment, one or more compounds of the present invention are administered with one or more additional therapeutic agents selected from an HCV protease inhibitor, an HCV polymerase inhibitor, an HCV replication inhibitor, a nucleoside and ribavirin. The combination therapies can include any combination of these additional therapeutic agents.

In another embodiment, one or more compounds of the present invention are administered with one additional therapeutic agent selected from an HCV protease inhibitor and ribavirin.

In still another embodiment, one or more compounds of the present invention are administered with two additional therapeutic agents selected from an HCV protease inhibitor, an HCV replication inhibitor, a nucleoside and ribavirin.

In another embodiment, one or more compounds of the present invention are administered with an HCV protease inhibitor and ribavirin. In another specific embodiment, one or more compounds of the present invention are administered with ribavirin.

In another embodiment, one or more compounds of the present invention are administered with three additional therapeutic agents selected from an HCV protease inhibitor, an HCV replication inhibitor, a nucleoside, a pegylated interferon and ribavirin.

In one embodiment, one or more compounds of the present invention are administered with one or more additional therapeutic agents selected from an HCV polymerase inhibitor, a viral protease inhibitor, and a viral replication inhibitor. In another embodiment, one or more compounds of the present invention are administered with one or more additional therapeutic agents selected from an HCV polymerase inhibitor, a viral protease inhibitor, and a viral replication inhibitor. In another embodiment, one or more compounds of the present invention are administered with one or more additional therapeutic agents selected from an HCV polymerase inhibitor, a viral protease inhibitor, and ribavirin.

In one embodiment, one or more compounds of the present invention are administered with one additional therapeutic agent selected from an HCV polymerase inhibitor, a viral protease inhibitor, and a viral replication inhibitor. In another embodiment, one or more compounds of the present invention are administered with ribavirin.

In one embodiment, one or more compounds of the present invention are administered with two additional therapeutic agents selected from an HCV polymerase inhibitor, a viral protease inhibitor, and a viral replication inhibitor.

In another embodiment, one or more compounds of the present invention are administered with ribavirin and another therapeutic agent.

In another embodiment, one or more compounds of the present invention are administered with ribavirin and another therapeutic agent, wherein the additional therapeutic agent is selected from an HCV polymerase inhibitor, a viral protease inhibitor, and a viral replication inhibitor.

In still another embodiment, one or more compounds of the present invention are administered with ribavirin and a viral protease inhibitor.

In another embodiment, one or more compounds of the present invention are administered with ribavirin and an HCV protease inhibitor.

In another embodiment, one or more compounds of the present invention are administered with ribavirin and either boceprevir or telaprevir.

In a further embodiment, one or more compounds of the present invention are administered with ribavirin and an HCV polymerase inhibitor.

In another embodiment, one or more compounds of the present invention are administered with ribavirin.

In one embodiment, one or more compounds of the present invention are administered with MK-5172.

In one embodiment, one or more compounds of the present invention are administered with sofosbuvir.

### Compositions and Administration

Due to their activity, the Silane-Containing Heterocyclic Compounds may be useful in veterinary and human medicine. As described above, the Silane-Containing Heterocyclic Compounds may be useful for treating or preventing HCV infection in a patient in need thereof.

When administered to a patient, the Silane-Containing Heterocyclic Compounds can be administered as a component of a composition that comprises a pharmaceutically acceptable carrier or vehicle. The present invention provides pharmaceutical compositions comprising an effective amount of at least one Silane-Containing Heterocyclic Compound and a pharmaceutically acceptable carrier. In the pharmaceutical compositions and methods of the present invention, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Solid form preparations include powders, tablets, dispersible granules, capsules, cachets and suppositories. Powders and tablets may be comprised of from about 0.5 to about 95 percent inventive composition. Tablets, powders, cachets and capsules can be used as solid dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum, and the like. Sweetening and flavoring agents and preservatives may also be included where appropriate.

Liquid form preparations include solutions, suspensions and emulsions and may include water or water-propylene glycol solutions for parenteral injection.

Also included are solid form preparations which are intended to be converted, shortly before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Additionally, the compositions of the present invention may be formulated in sustained release form to provide the rate controlled release of any one or more of the components or active ingredients to optimize therapeutic effects, i.e., antiviral activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In one embodiment, the one or more Silane-Containing Heterocyclic Compounds are administered orally.

In another embodiment, the one or more Silane-Containing Heterocyclic Compounds are administered intravenously.

In still another embodiment, the one or more Silane-Containing Heterocyclic Compounds are administered sublingually.

In one embodiment, a pharmaceutical preparation comprising at least one Silane-Containing Heterocyclic Compound is in unit dosage form. In such form, the preparation is subdivided into unit doses containing effective amounts of the active components.

Compositions can be prepared according to conventional mixing, granulating or coating methods, respectively, and the present compositions can contain, in one embodiment, from about 0.1% to about 99% of the Silane-Containing Heterocyclic Compound(s) by weight or volume. In various embodiments, the present compositions can contain, in one embodiment, from about 1% to about 70% or from about 5% to about 60% of the Silane-Containing Heterocyclic Compound(s) by weight or volume.

The amount and frequency of administration of the Silane-Containing Heterocyclic Compounds will be regulated according to the judgment of the attending clinician considering such factors as age, condition and size of the patient as well as severity of the symptoms being treated. Generally, a total daily dosage of the at least one Silane-Containing Heterocyclic Compound(s) alone, or when administered as combination therapy, can range from about 1 to about 2500 mg per day, although variations will necessarily occur depending on the target of therapy, the patient and the route of administration. In one embodiment, the dosage is from about 10 to about 1000 mg/day, administered in a single dose or in 2-4 divided doses. In another embodiment, the dosage is from about 1 to about 500 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In another embodiment, the dosage is from about 500 to about 1500 mg/day, administered in a single dose or in 2-4 divided doses. In still another embodiment, the dosage is from about 500 to about 1000 mg/day, administered in a single dose or in 2-4 divided doses. In yet another embodiment, the dosage is from about 100 to about 500 mg/day, administered in a single dose or in 2-4 divided doses.

The compositions of the invention can further comprise one or more additional therapeutic agents, selected from those listed above herein. Accordingly, in one embodiment, the present invention provides compositions comprising: (i) at least one Silane-Containing Heterocyclic Compound or a pharmaceutically acceptable salt thereof; (ii) one or more additional therapeutic agents that are not a Silane-Containing Heterocyclic Compound; and (iii) a pharmaceutically acceptable carrier, wherein the amounts in the composition are together effective to treat HCV infection.

In one embodiment, the present invention provides compositions comprising a Compound of Formula (I) and a pharmaceutically acceptable carrier.

In another embodiment, the present invention provides compositions comprising a Compound of Formula (I), a pharmaceutically acceptable carrier, and a second therapeutic agent selected from the group consisting of HCV antiviral agents, immunomodulators, and anti-infective agents.

In another embodiment, the present invention provides compositions comprising a Compound of Formula (I), a pharmaceutically acceptable carrier, and to additional therapeutic agents, each of which are independently selected from the group consisting of HCV antiviral agents, immunomodulators, and anti-infective agents.

### Kits

In one aspect, the present invention provides a kit comprising a therapeutically effective amount of at least one Silane-Containing Heterocyclic Compound, or a pharmaceutically acceptable salt, solvate, ester or prodrug of said compound and a pharmaceutically acceptable carrier, vehicle or diluent.

In another aspect the present invention provides a kit comprising an amount of at least one Silane-Containing Heterocyclic Compound, or a pharmaceutically acceptable salt, solvate, ester or prodrug of said compound and an amount of at least one additional therapeutic agent listed above, wherein the amounts of the two or more active ingredients result in a desired therapeutic effect. In one embodiment, the one or more Silane-Containing Heterocyclic Compounds and the one or more additional therapeutic agents are provided in the same container. In one embodiment, the one or more Silane-Containing Heterocyclic Compounds and the one or more additional therapeutic agents are provided in separate containers.

Methods for Making the Compounds of Formula (I)

The Compounds of Formula (I) may be prepared from known or readily prepared starting materials, following methods known to one skilled in the art of organic synthesis. Methods useful for making the Compounds of Formula (I) are set forth in the Examples below and generalized in Schemes 1-4 below. Alternative synthetic pathways and analogous structures will be apparent to those skilled in the art of organic synthesis.

Scheme 1 shows methods useful for making the compounds of formula G3, which may be useful intermediates for making the Compounds of Formula (I).

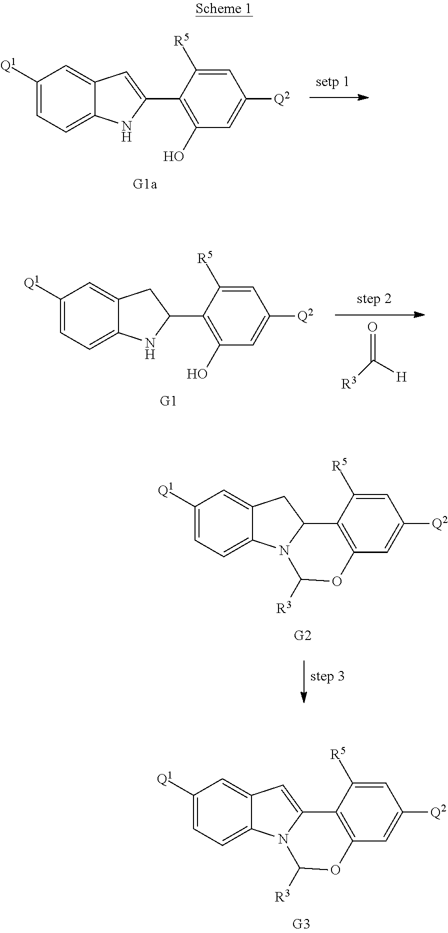

Wherein R³ and R⁵ are defined above for the Compounds of Formula (I) and Q¹ and Q² are each independently halo, hydroxyl, or a protected hydroxyl group, such as a methoxy or benzyloxy group.

An indole compound of formula G1a (which can be prepared as described in PCT International Publication No. WO 2012/040923) can be treated with tin in conc.HCl/EtOH solution to provide compounds of formula G1. A compound of formula G1 can be reacted with an aldehyde of formula R³CHO in the presence of an acid to provide tetracyclic compounds of formula G2. Compounds of formula G2 can then be oxidized to provide the tetracyclic compounds of formula G3.

Scheme 2 shows methods useful for making the compounds of formula G5, which may be useful intermediates for making the Compounds of Formula (I).

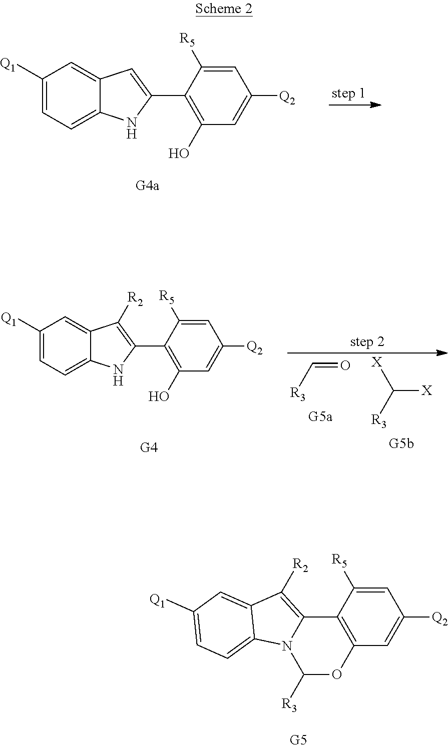

Wherein R², R³ and R⁵ are defined above for the Compounds of Formula (I), X is halo, and Q¹ and Q² are each independently halo, hydroxyl, or a protected hydroxyl group, such as a methoxy or benzyloxy group.

A compound of formula G4a (which can be prepared as described in PCT International Publication No. WO 2012/040923) can be halogenated to provide the compounds of formula G4. A compounds of formula G4 can then be converted to the compounds of formula G5 via reaction with an aldehyde of formula G5a in the presence of an acid, or alternatively, by reaction with a dihalo compound of formula G5b in the presence of a base.

Scheme 3 shows methods useful for making the compounds of formula G12, which may be useful intermediates for making the Compounds of Formula (I).

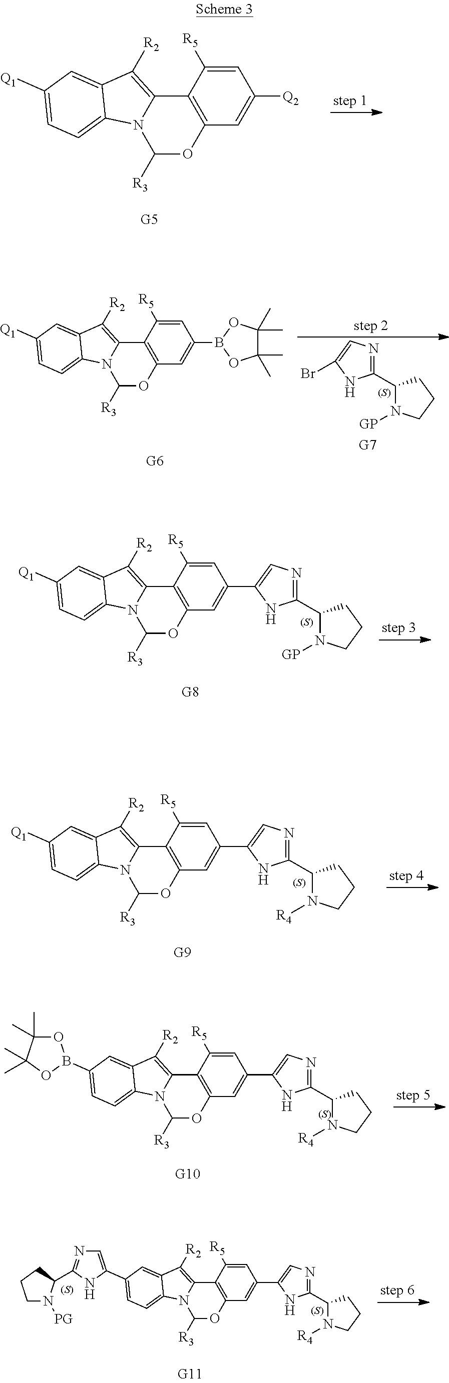

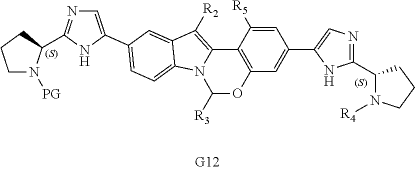

Wherein R², R³, R⁴ and R⁵ are defined above for the Compounds of Formula (I), PG is a secondary amino protecting group, and Q¹ and Q² are each independently halo, hydroxyl, or a protected hydroxyl group, such as a methoxy or benzyloxy group.

A compound of formula G5 can be reacted with bis(pinacolato)diboron to provide the compounds of formula G6. A compound of formula G6 can then undergo a Pd-mediated coupling with a bromo compound of formula G7 (prepared as described in PCT International Publication No. WO 2012/040923) to provide the compounds of formula G8. Compounds of formula G8 can then be deprotected and subjected to an amide coupling with a desired cap compound to provide a compound of formula G9. A compound of formula G9 is then subjected to a Pd-mediated coupling with bis(pinacolato)diboron to provide the boronic ester compounds of formula G10. A compound of formula G10 can then undergo a Pd-mediated coupling with a bromo compound of formula G7 (prepared as described in PCT International Publication No. WO 2012/040923) to provide the compounds of formula G11. Compounds of formula G11 can then be deprotected and subjected to an amide coupling with a desired cap compound to provide a compound of formula G12. Distereoisomers of the synthetic intermediates and final products can be separated using SFC or HPLC with chiral columns.

Scheme 4 shows methods useful for making the compounds of formula G18, which correspond to the Compounds of Formula (I).

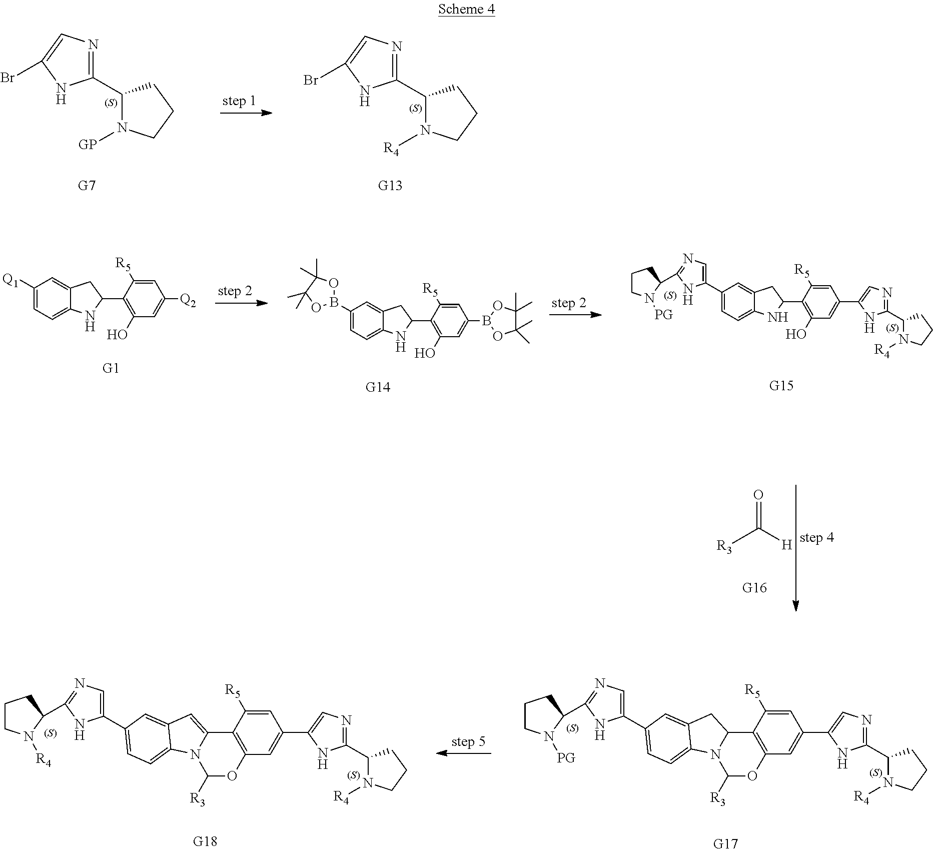

Wherein R³, R⁴ and R⁵ are defined above for the Compounds of Formula (I), PG is a secondary amino protecting group, and Q¹ and Q² are each independently halo, hydroxyl, or a protected hydroxyl group, such as a methoxy or benzyloxy group.

A compounds of formula G7 can then be deprotected and subjected to an amide coupling with a desired cap compound to provide a compound of formula G12. A compound of formula G1 can be converted to compound of formula G14 via a Pd mediated coupling reaction with bis(pinacolato)diboron. A compound of formula G14 can then be subjected to a Pd-mediated coupling with 2 equivalents of G13 to provide the compounds of formula G15. A compound of formula G15 can then be converted to the compounds of formula G17 via reaction with an aldehyde of formula G16 in the presence of an acid. Compounds of formula G17 can then be oxidized to provide the tetracyclic compounds of formula G18. Distereoisomers of G18 can be reparated by SFC using chiral columns.

In some of the Compounds of Formula (I) contemplated in Schemes 1-4, amino acids (such as, but not limited to proline, 4-(R)-fluoroproline, 4-(S)-fluoroproline, 4,4-difluoroproline, 4,4-dimethylsilylproline, aza-bicyclo[2.2.1]heptane carboxylic acid, aza-bicyclo[2.2.2]octane carboxylic acid, (S)-2-piperidine carboxylic acid, valine, alanine, norvaline, etc) are incorporated as part of the structures. Methods have been described in the organic chemistry literature as well as in Banchard US 2009/0068140 (Published Mar. 9, 2009) for the preparation of such amino acid-derived intermediates.

One skilled in the art of organic synthesis will recognize that the synthesis of fused tetracyclic cores contained in Compounds of Formula (I) may require protection of certain functional groups (i.e., derivatization for the purpose of chemical compatibility with a particular reaction condition). Suitable protecting groups for the various functional groups of these Compounds and methods for their installation and removal are well known in the art of organic chemistry. A summary of many of these methods can be found in Greene et al., Protective Groups in Organic Synthesis, Wiley-Interscience, New York, (1999).

One skilled in the art of organic synthesis will also recognize that one route for the synthesis of the fused tetracyclic cores of the Compounds of Formula (I) may be more desirable depending on the choice of appendage substituents. Additionally, one skilled in the art will recognize that in some cases the order of reactions may differ from that presented herein to avoid functional group incompatibilities and thus adjust the synthetic route accordingly.

One skilled in the art of organic synthesis will recognize that the synthesis of certain fused tetracyclic cores of the Compounds of Formula (I) require the construction of an amide bond. Methods useful for making such amide bonds, include but are not limited to, the use of a reactive carboxy derivative (e.g., an acid halide, or ester at elevated temperatures) or the use of an acid with a coupling reagent (e.g., HOBt, EDCI, DCC, HATU, PyBrop) with an amine.

The preparation of multicyclic intermediates useful for making the fused tetracyclic ring systems of the Compounds of Formula (I) have been described in the literature and in compendia such as “Comprehensive Heterocyclic Chemistry” editions I, II and III, published by Elsevier and edited by A. R. Katritzky & R. JK Taylor. Manipulation of the required substitution patterns have also been described in the available chemical literature as summarized in compendia such as “Comprehensive Organic Chemistry” published by Elsevier and edited by DH R. Barton and W. D. Ollis; “Comprehensive Organic Functional Group Transformations” edited by edited by A. R. Katritzky & R. JK Taylor and “Comprehensive Organic Transformation” published by Wily-CVH and edited by R. C. Larock.

The Compounds Formula (I) may contain one or more silicon atoms. The Compounds contemplated in this invention in general can be prepared using the carba-analog methodology unless otherwise noted. A recent review of the synthesis of silicon containing Compounds can be found in “Silicon Chemistry: from Atom to Extended Systems”, Ed P. Jutzi & U. Schubet; ISBN 978-3-527-30647-3. Preparation of silyl containing amino acids has been described. See Bolm et al., Angew. Chem. Int Ed., 39:2289 (2000). Descriptions of improved cellular update (Giralt, J. Am. Chem. Soc., 128:8479 (2006)) and reduced metabolic processing of silyl containing Compounds have been described (Johansson et al., Drug Metabolism & Disposition, 38:73 (2009)).

The starting materials used and the intermediates prepared using the methods set forth in Schemes 1-5 may be isolated and purified if desired using conventional techniques, including but not limited to filtration, distillation, crystallization, chromatography and alike. Such materials can be characterized using conventional means, including physical constants and spectral data.

One skilled in the art will be aware of standard formulation techniques as set forth in the open literature as well as in textbooks such as Zheng, “Formulation and Analaytical Development for Low-dose Oral Drug Products”, Wiley, 2009, ISBN.

### EXAMPLES

### General Methods

Solvents, reagents, and intermediates that are commercially available were used as received. Reagents and intermediates that are not commercially available were prepared in the manner as described below. ¹H NMR spectra were obtained on a Bruker Avance 500 (500 MHz) and are reported as ppm downfield from Me4Si with number of protons, multiplicities, and coupling constants in Hertz indicated parenthetically. Where LC/MS data are presented, analyses was performed using an Applied Biosystems API-100 mass spectrometer and Shimadzu SCL-10A LC column: Altech platinum C18, 3 micron, 33 mm×7 mm ID; gradient flow: 0 minutes—10% CH₃CN, 5 minutes—95% CH₃CN, 5-7 minutes—95% CH₃CN, 7 minutes—stop. The retention time and observed parent ion are given. Flash column chromatography was performed using pre-packed normal phase silica from Biotage, Inc. or bulk silica from Fisher Scientific. Unless otherwise indicated, column chromatography was performed using a gradient elution of hexanes/ethyl acetate, from 100% hexanes to 100% ethyl acetate.

### Example 1

### Preparation of Intermediate Compound Int-1

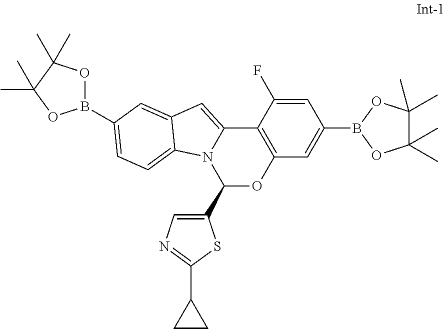

Int-1 was made using the method described in Example 12 of PCT International Patent Publication No. WO 2014/110687.

### Example 2

### Preparation of Intermediate Compound Int-2

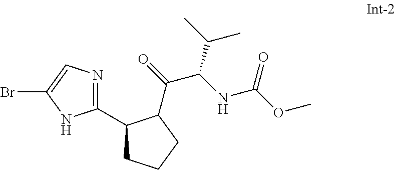

Compound Int-2 was made using the method described in Example 7 of PCT International Patent Publication No. WO 2012/040923.

### Example 3

### Preparation of Intermediate Compound Int-3

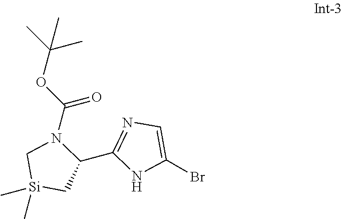 [SMILES: CC(C)(C)OC(=O)N1C[Si](C)(C)C[C@H]1c1ncc(Br)[nH]1]

Compound Int-3 was made using the method described in Example 8 of PCT International Patent Publication No. WO 2012/122716.

### Example 4

### Preparation of Intermediate Compound Int-4

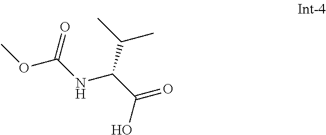

Compound Int-4 was made using the method described in Example 1 of PCT International Patent Publication No. WO 2013/039876.

### Example 5

### Preparation of Intermediate Compound Int-5

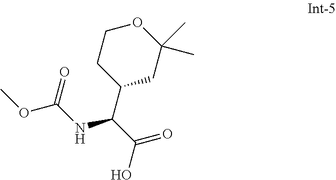

Compound Int-5 was made using the method described in Example 7 of PCT International Patent Publication No. WO 2014/110705.

### Example 6

### Preparation of Intermediate Compound Int-6

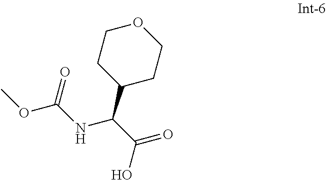

Compound Int-6 was made using the method described in Example 4 of PCT International Patent Publication No. WO 2012/040923.

### Example 7

### Preparation of Compound 1

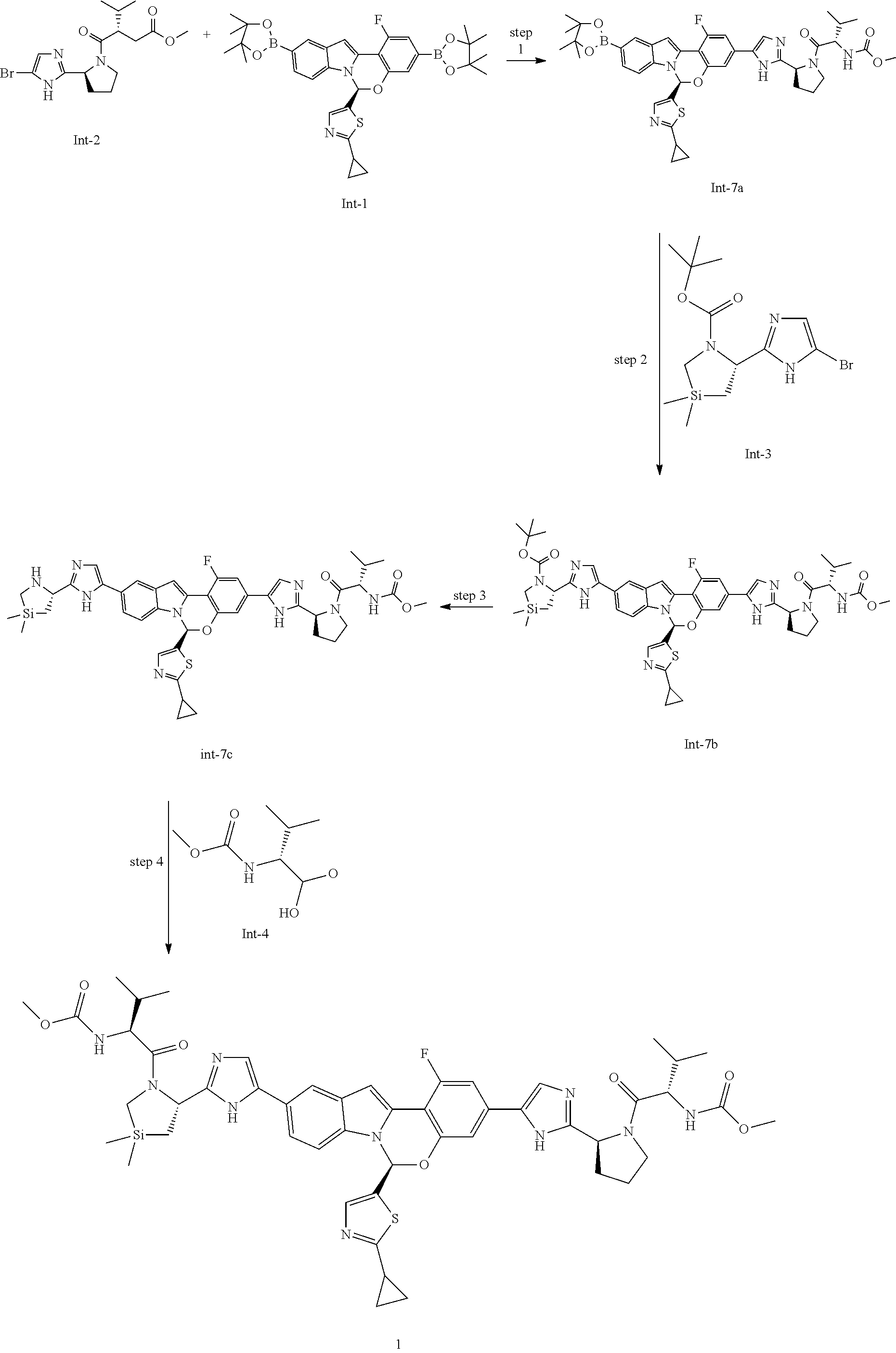

### Step 1 Synthesis of Compound Int-7a

To a 100 mL round bottom flask was added Int-2 (1215 mg, 3.26 mmol), Int-1 (2000 mg, 3.26 mmol), and PdCl₂(dppf)-CH₂Cl₂ adduct (133 mg, 0.163 mmol, purchased from Sigma Aldrich). The flask was degassed under vacuum then put under nitrogen atmosphere. Dioxane (3.26E+04 μl) and potassium carbonate (4069 μl, 8.14 mmol) was added and the reaction was allowed to stir at 80° C. for 5 hours. The aqeuous layer was separated and extracted with EtOAc (5 mL). The organic layers were combined and dried over anhydrous Na₂SO₄, then concentrated in vacuo and the resulting residue was purified using SiO₂ chromatography (80 g, Hexane/EtOAc, 30% to 100%) to provide compound Int-7a (1.36 g, 1.742 mmol). ¹H NMR (500 MHz, methanol-d4) δ: 7.96 (d, J=10 Hz, 2H), 7.87 (s, 1H), 7.70 (s, 1H), 7.57 (m, 2H), 7.41 (d, J=15 Hz, 1H), 7.28 (s, 1H), 7.16 (m, 1H), 7.10 (s, 1H), 5.39 (t, J=10 Hz, 1H), 5.20 (t, J=10 Hz, 1H), 4.53 (d, J=5 Hz, 1H), 4.23 (d, J=5 Hz, 1H), 4.08 (m, 1H), 3.88 (m, 1H), 3.65 (s, 3H), 3.64 (s, 3H), 3.52 (d, J=20 Hz, 1H), 3.16 (d, J=15 Hz, 1H), 2.50 (m, 1H), 2.27-2.05 (m, 8H), 1.72 (dd, J=10, 20 Hz, 1H), 1.24 (dd, J=10, 20 Hz, 1H), 1.08 (m, 2H), 0.99-0.85 (m, 14H), 0.44 (s, 3H), 0.34 (s, 3H). LC/MS: Anal. Calcd. For [M+H]⁺ C41H46BFN6O6S: 781.7; found 781.4.

### Step 2-Synthesis of Compound Int-7b

To a 8 mL vial was added Int-3 (346 mg, 0.959 mmol), compound Int-7a (624 mg, 0.799 mmol), and PdCl₂(dppf)-CH₂Cl₂ adduct (65.3 mg, 0.080 mmol, purchased from Sigma Aldrich). The vial was degassed under vacuum then put under nitrogen atmosphere. Dioxane (7993 μl) and potassium carbonate (1199 μl, 2.398 mmol) was added. The reaction was allowed to stir at 80° C. for 5 hours. The aqueous layer was separated and extracted with EtOAc (5 mL). The organic layers were combined and dried over anhydrous Na₂SO₄. The reaction mixture was concentrated in vacuo and the resulting residue was purified using SiO₂ chromatography (80 g, Hexane/EtOAc, 30% to 100%) to provide Int-7b (614 mg). LC/MS: Anal. Calcd. For [(M+2H)/2]⁺ C48H56FN9O6SSi: 467.7; found 467.9.

### Step 3-Synthesis of Compound Int-7c

To a 40 mL pressure vial with pressure release cap was added Int-7b (564 mg, 0.604 mmol), CH₂Cl₂ (3019 μl), MeOH (3019 μl), and HCl (1509 μl, 6.04 mmol). The reaction was allowed to stir at 25° C. for 2 hours, then the reaction mixture was concentrated in vacuo and the resulting residue was dried under vacuum for 2 hours to provide Int-7c as its tris HCl salt (570 mg), which was used without further purification. LC/MS: Anal. Calcd. For [(M+2H)/2]⁺ C43H48FN9O4SSi: 417.7; found 417.9.

### Step 4-Synthesis of Compound 1

To a 8 mL pressure vial with pressure release cap was added Int-7c (50 mg, 0.060 mmol), Int-4 (13.65 mg, 0.078 mmol), HATU (34.2 mg, 0.090 mmol), and DMF (599 μl). The reaction was allowed to stir at room temperature for 2 minutes, then was cooled to 0° C. and DIPEA was added. The reaction mixture was allowed to stir at 0° C. for 1 hour, then water (0.3 mL) and TFA (0.3 mL) were added. The solution was allowed to stir at room temperature for 30 minutes, and then the product was purified using a C18 column (50 g, CH₃CN/water 10% to 70%, with 0.05% TFA) to provide compound 1 (45.9 mg). 1H-NMR (500 Mz), LC/MS: Anal. Calcd. For [(M+2H)/2]⁺ C50H59FN1007SSi: 496.2; found 496.4.

Compounds 2-5, depicted in the table below, were made using the methods described in Example 7 and substituting the appropriate reactants and/or reagents.

| | | Ob- |
|---|---|---|
| Com- | | served |
| pound | | [(M + |
| ID | Structure | 2H)/2]⁺ |
| 2 | 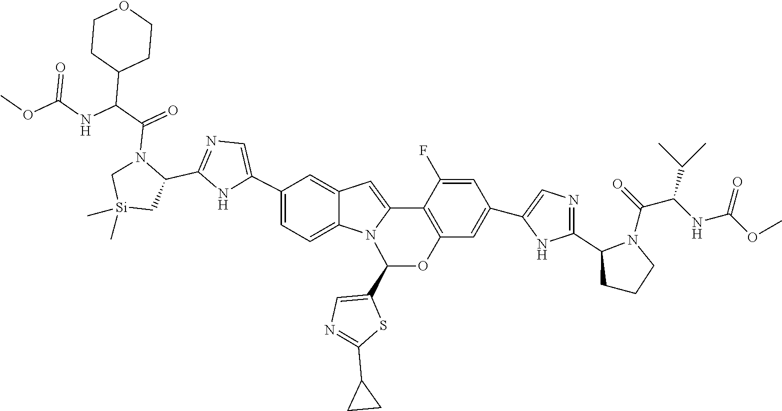 | 517.1 |
| 3 | 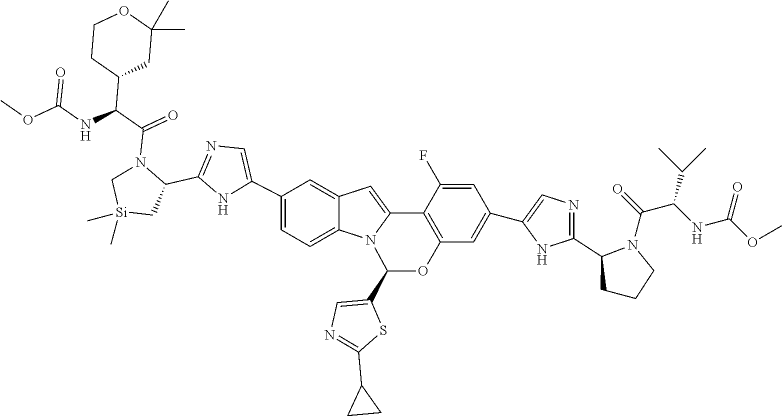 | 531.5 |
| 4 | 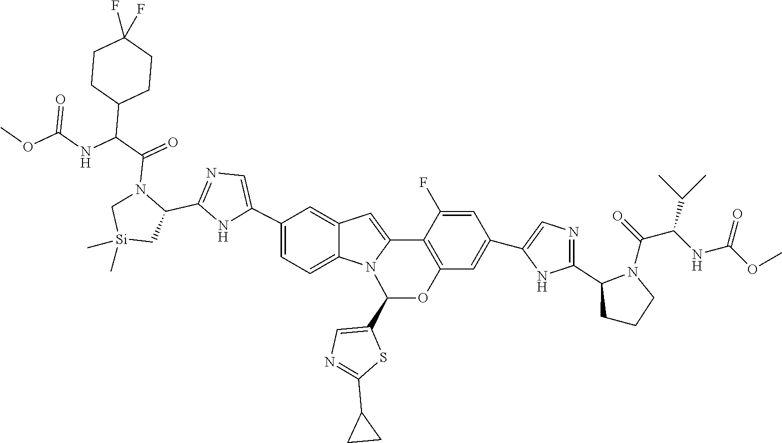 | 543.6 |
| 5 | 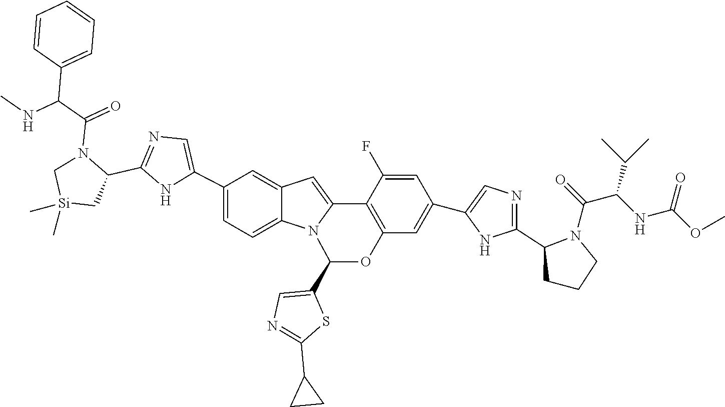 | 491.1 |

### Example 8

### Preparation of Compound 6

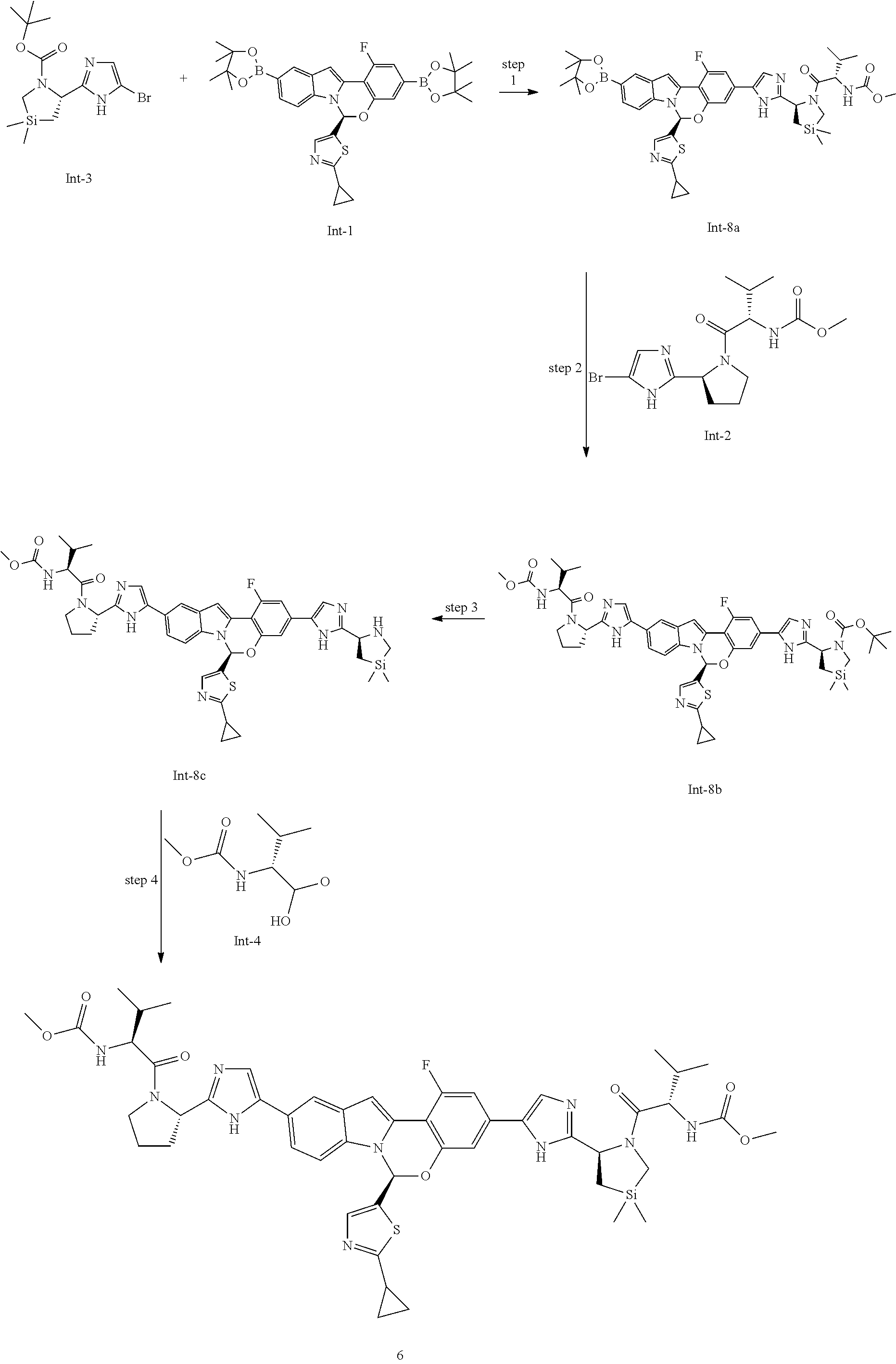

### Step 1-Synthesis of Compound Int-8a

To a 100 mL round bottom flask was added Int-1 (1000 mg, 1.628 mmol), and PdCl₂(dppf)-CH₂Cl₂ adduct (66.5 mg, 0.081 mmol, purchased from Sigma Aldrich), and Int-3 (587 mg, 1.628 mmol). The flask was degassed under vacuum then put under nitrogen atmosphere. Dioxane (1.63E+04 μl) and potassium carbonate (2035 μl, 4.07 mmol) was added. The reaction was allowed to stir at 80° C. for 5 hours. The aqueous layer was separated and extracted with EtOAc (5 mL). The organic layers were combined and dried over anhydrous Na₂SO₄. The solution was concentrated in vacuo and the resulting residue was purified using SiO₂ chromatography (80 g, Hexane/EtOAc, 30% to 100%) to provide Int-8a (880 mg). LC/MS: Anal. Calcd. For [M+H]⁺ C40H47BFN505SSi: 767.3; found 768.5.

### Step 2-Synthesis of Compound Int-8b

To a 250 mL flask was added Int-2 (496 mg, 1.328 mmol), Int-8a (850 mg, 1.107 mmol), and PdCl₂(dppf)-CH₂Cl₂ adduct (90 mg, 0.111 mmol, purchased from Sigma Aldrich). The flask was degassed under vacuum then put under nitrogen atmosphere. Dioxane (11 mL) and potassium carbonate (1661 μl, 3.32 mmol) were added and the reaction was allowed to stir at 80° C. for 16 hours. The aqueous layer was separated and extracted with EtOAc (50 mL) and the organic layers were combined and dried over anhydrous Na₂SO₄. The reaction mixture was concentrated in vacuo and the resulting residue was purified using flash column chromatography on silica gel (80 g, Hexane/EtOAc, 30% to 100%) to provide Int-8b (502 mg). LC/MS: Anal. Calcd. For [(M+2H)/2]⁺ C48H56FN9O6SSi: 467.7; found 468.0.

### Step 3-Synthesis of Compound Int-8c

To a 40 mL pressure vial with pressure release cap was added Int-8b (490 mg, 0.525 mmol), CH₂Cl₂ (9537 μl), MeOH (954 μl), and HCl (1967 μl, 7.87 mmol). The reaction was allowed to stir at 25° C. for 8 hours, then the reaction mixture was concentrated in vacuo and the resulting residue was dried under vacuum for 16 hours to provide Int-8c as its tris HCl salt, which was used without further purification. (495 mg). LC/MS: Anal. Calcd. For [(M+2H)/2]⁺ C43H48FN9O4SSi: 417.7; found 417.6.

### Step 4-Synthesis of Compound 6

To a 4 mL pressure vial with pressure release cap was added Int-4 (5.57 mg, 0.032 mmol), HOBT (5.84 mg, 0.038 mmol), EDC (7.32 mg, 0.038 mmol) and Int-8c tris HCl salt (30 mg, 0.032 mmol), and DMF (318 μl). The reaction mixture was allowed to stir at 25° C. for 5 minutes, then DIPEA (33.3 μl, 0.191 mmol) was added and the reaction was allowed to stir at 25° C. for 5 hours. TFA (0.1 mL) was then added and the reaction was allowed to stir at 25° C. for 30 minutes. The reaction mixture was then diluted with DMSO to 1.5 mL and the resulting solution was directly purified using HPLC (Column: Xbridge C18 5 um 19×50 mm; Flow rate: 25 mL/min; Temperature: 25 C; Mobile phase: 17% ACN (0.1% NH₄OH) to 90% over 8 min; Detector: UV 215 nm) to provide compound 6 (12.7 mg). ¹H NMR (500 MHz, METHANOL-d4) δ: 7.89 (d, J=5 Hz, 1H), 7.80 (s, 1H), 7.51 (d, J=10 Hz, 1H), 7.38 (d, J=10 Hz, 1H), 7.29 (d, J=10 Hz, 1H), 7.20 (s, 1H), 7.09 (s, 1H), 6.99 (d, J=5 Hz, 1H), 5.79 (t, J=5 Hz, 1H), 5.18 (t, J=5 Hz, 1H), 4.53 (d, J=10 Hz, 1H), 4.23 (d, J=10 Hz, 1H), 3.98 (m, 1H), 3.87 (m, 1H), 3.65 (s, 6H), 3.38 (d, J=20 Hz, 1H), 3.06 (d, J=20 Hz, 1H), 2.40-2.05 (m, 8H), 1.34 (d, J=10, 2H), 1.06-0.86 (m, 15H), 0.30 (s, 3H), 0.23 (d, 3H). LC/MS: Anal. Calcd. For [(M+2H)/2]⁺ C50H59FN10O7SSi: 496.2; found 496.5. Separation method: Xbridge C18 5 um 19×50 mm, 25 mL/min, 25 C, 17% ACN (0.1% NH₄OH) to 90% over 8 min, UV 215 nm.

Compounds 7-11, depicted in the table below, were made using the methods described in Example 8 and substituting the appropriate reactants and/or reagents.

| | | Ob- |
|---|---|---|
| Com- | | served |
| pound | | [(M + |
| No. | Structure | 2H)/2]⁺ |
| 7 | 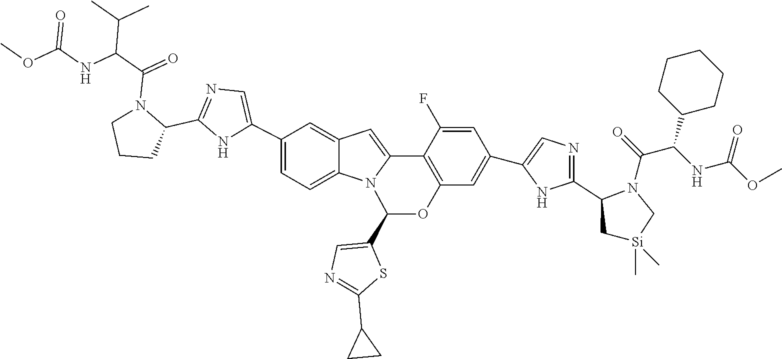 | 517.6 |
| 8 | 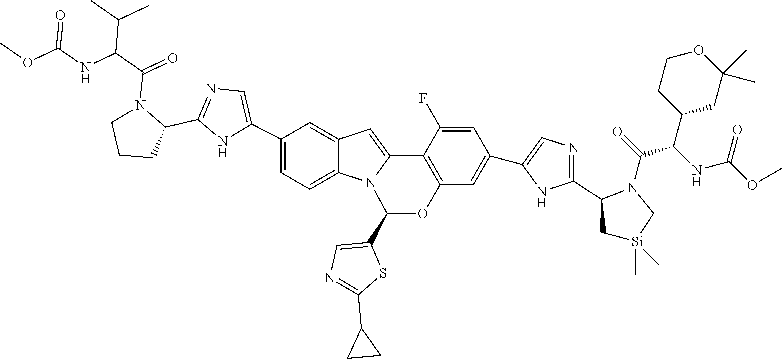 | 531.5 |
| 9 | 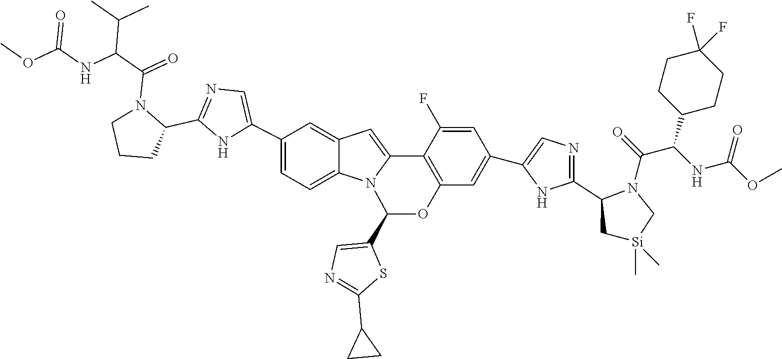 | 534.5 |
| 10 | 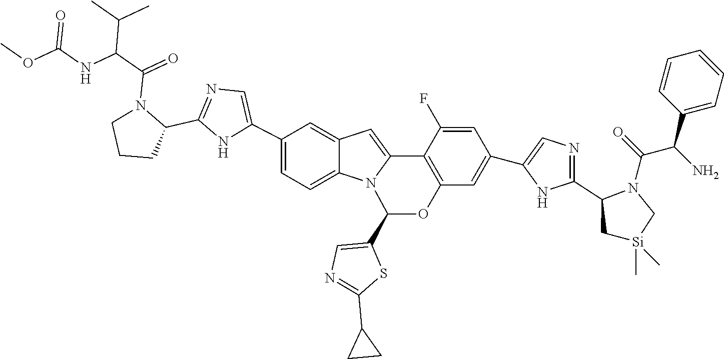 [SMILES: COC(=O)NC(C(=O)N1CCC[C@H]1c1ncc(-c2ccc3c(c2)cc2n3[C@H](c3cnc(C4CC4)s3)Oc3cc(-c4cnc([C@@H]5C[Si](C)(C)CN5C(=O)[C@H](N)c5ccccc5)[nH]4)cc(F)c3-2)[nH]1)C(C)C] | 484.2 |
| 11 | 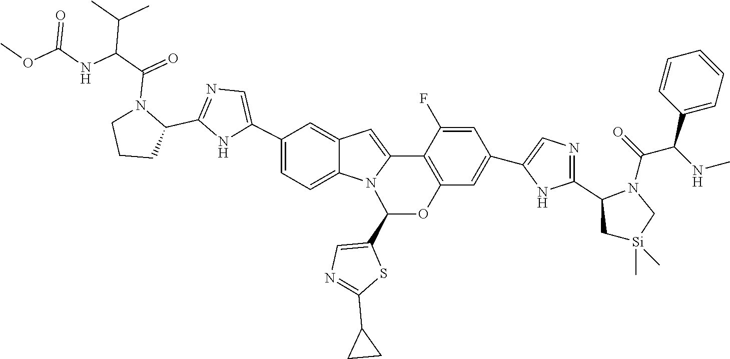 | 491.5 |

### Example 9

### Preparation of Compound 12

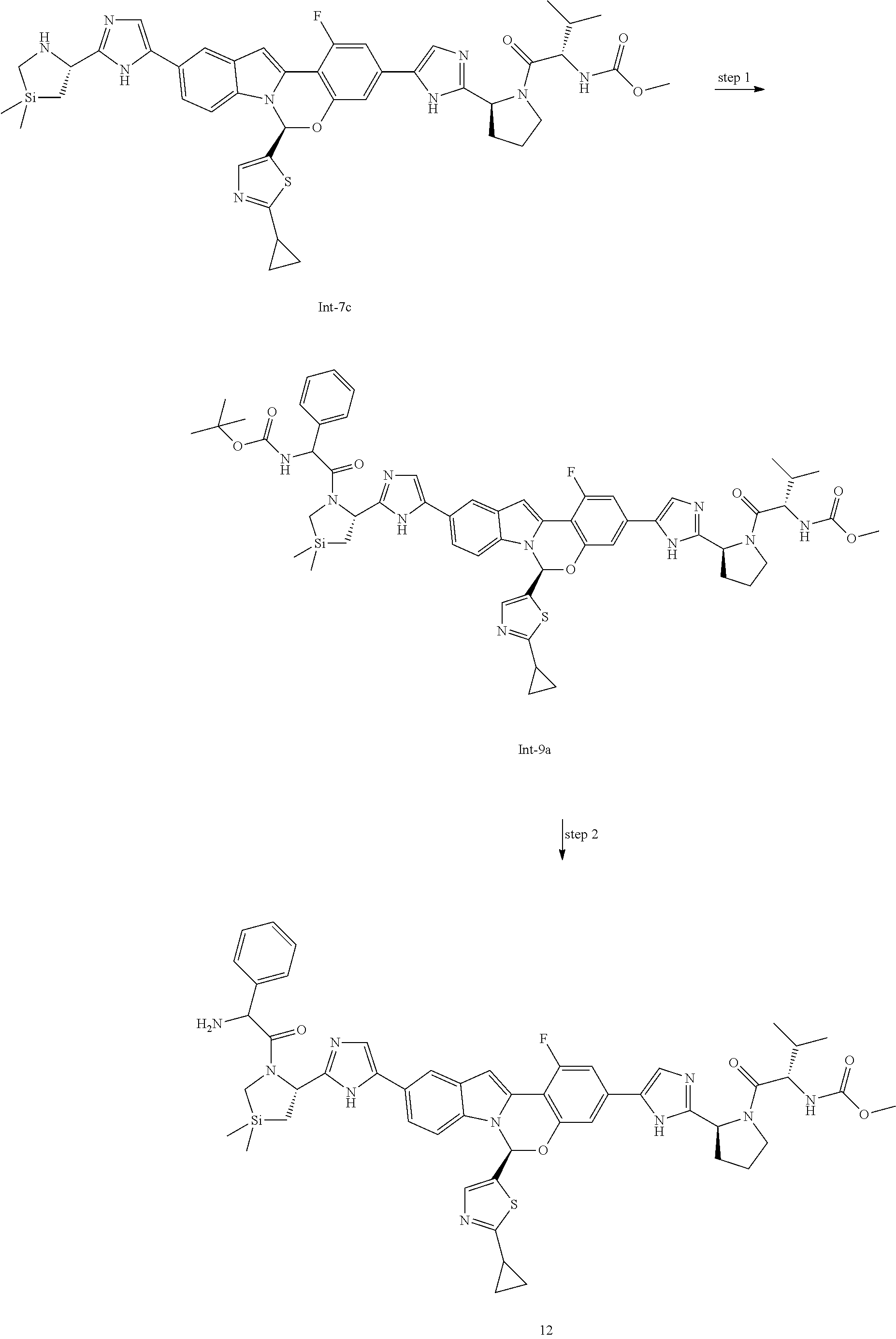

### Step 1-Synthesis of Compound Int-9a

To a 8 mL pressure vial with pressure release cap was added Int-7c (50 mg, 0.060 mmol), (R)-2-((tert-butoxycarbonyl)amino)-2-phenylacetic acid (15.06 mg, 0.060 mmol), EDC (13.79 mg, 0.072 mmol), and HOBT (11.02 mg, 0.072 mmol), and DMF (599 μl). The reaction was allowed to stir at room temperature for 2 minutes. DIPEA (0.0534 mL, 0.3 mmol) were added. The solution was allowed to stir at 25° C. for 4 hour. The reaction mixture was diluted with DMSO to 1.3 mL and the resulting residue was purified to provide Int-9a (35.9 mg). LC/MS: Anal. Calcd. For [M+H]⁺ C56H63FN10O7SSi: 1066.4; found 1066.4. Separation method: Xbridge C18 5 um 19×50 mm, 25 mL/min, 25 C, 17% ACN (0.1% NH₄OH) to 90% over 8 min, UV 215 nm.

### Step 2-Synthesis of Compound 12

Int-9a (35.9 mg, 0.034 mmol) was dissolved in CH₂Cl₂ (1 mL) and HCl (4M in Dioxane 0.2 mL) was added. The reaction was allowed to stir at 25° C. for 5 hours, then the reaction mixture was concentrated in vacuo for 16 hours to provide compound 12 as its HCl salt (36.5 mg). ¹H NMR (500 MHz, METHANOL-d4) δ: 8.01 (d, J=5 Hz, 2H), 7.78 (d, J=3 Hz 1H), 7.66 (m, 1H), 7.53 (m, 2H), 7.48 (m, 2H), 7.39 (t, J=7.5 Hz 1H), 7.28 (dd, J=5, 10 Hz 1H), 7.12 (s, 1H), 5.68 (dd, J=5.10 Hz, 1H), 5.60 (d, J=20 Hz, 1H), 5.52 (t, J=10 Hz, 1H), 5.22 (t, J=10 Hz, 1H), 4.21 (d, J=10 Hz, 1H), 4.10 (m, 1H), 3.88 (m, 1H), 3.74 (t, J=5 Hz, 1H), 3.67 (s, 3H), 3.59 (m, 1H), 3.34 (d, J=10 Hz, 1H), 2.67 (d, J=17 Hz, 1H), 2.58 (d, J=17 Hz, 1H), 2.35 (m, 1H), 2.19 (m, 2H), 2.05 (m, 1H), 1.75 (dd, J=10, 20 Hz, 1H), 1.66 (dd, J=10, 20 Hz, 1H), 1.35 (dd, J=10, 20 Hz, 1H), 1.17 (dd, J=5, 20 Hz, 1H), 1.08 (m, 2H), 0.98 (m, 1H), 0.93-0.89 (m, 7H), 0.37 (d, J=5 Hz, 3H), 0.26 (s, 3H). LC/MS: Anal. Calcd. For [(M+2H)/2]⁺ C₄₄H₄₉FN₈O₄SSi: 417.4; found 417.4.

### Example 10

### Preparation of Compound 13

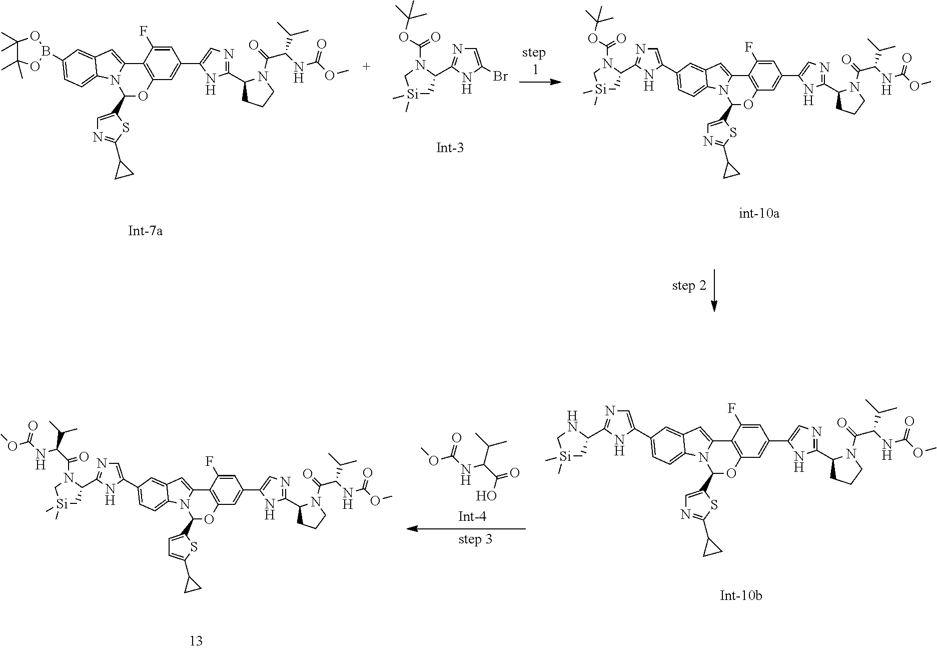

### Step 1-Synthesis of Compound Int-10a

To a 100 mL flask was added Int-3 (261 mg, 0.723 mmol), Int-7a (470 mg, 0.603 mmol), potassium carbonate (2M in water, 0.904 mL, 1.808 mmol), and PdCl₂(dppf)-CH₂Cl₂ adduct (49.2 mg, 0.060 mmol, purchased from Sigma-Aldrich). The flask was degassed under vacuum then put under nitrogen atmosphere. Dioxane (6028 μl) and potassium carbonate (904 μl, 1.808 mmol) was added and the reaction was allowed to stir at 80° C. for 20 hours. The aqueous layer was separated and extracted with EtOAc (50 mL) and the organic layers were combined and dried over anhydrous Na₂SO₄. The solution was concentrated in vacuo and the residue obtained was purified using SiO₂ chromatography (40 g, Hexane/EtOAc, 30% to 100%) to provide Int-10a (412 mg). LC/MS: Anal. Calcd. For [(M+2H)/2]⁺ C49H57FN8O6SSi: 467.2; found 467.4.

### Step 2-Synthesis of Compound Int-10b

To a 40 mL pressure vial with pressure release cap was added Int-10a (334 mg, 0.358 mmol), CH₂Cl₂ (3254 μl), Dioxane (325 μl), and HCl (895 μl, 3.58 mmol). The reaction was allowed to stir at 25° C. for 4 hours, then the reaction mixture was concentrated and dried under vacuum for 16 hours to provide Int-10b as its tris HCl salt (337 mg), which was used without further purification. LC/MS: Anal. Calcd. For [(M+2H)/2]⁺ C44H49FN8O4SSi: 417.4; found 417.4.

### Step 3-Synthesis of Compound 13

To a 4 mL pressure vial with pressure release cap was added Int-4 (5.57 mg, 0.032 mmol), HOBT (5.84 mg, 0.038 mmol), EDC (7.32 mg, 0.038 mmol), Int-10b tris HCl salt (30 mg, 0.032 mmol), and DMF (318 μl). The reaction was allowed to stir at 25° C. for 5 minutes, then DIPEA (33.3 μl, 0.191 mmol) was added. The reaction was allowed to stir at 25° C. for 5 hours. TFA (0.1 mL) were added and the reaction was then allowed to stir at 25° C. for an additional 30 minutes. The reaction mixture was diluted with DMSO to a volume of 1.5 mL and the resulting residue was purified to provide compound 13 (12.7 mg). ¹H NMR (500 MHz, METHANOL-d4) δ: 7.56 (t, J=15 Hz, 1H), 7.47 (d, J=10 Hz 1H), 7.38 (d, J=10 Hz 1H), 7.24 (m, 1H), 7.17 (s, 1H), 6.95 (d, J=5 Hz 1H), 6.47 (d, J=5 Hz 1H), 6.38 (d, J=5 Hz, 1H), 5.80 (dd, J=5.10 Hz, 1H), 5.13 (t, J=5 Hz, 1H), 4.51 (d, J=10 Hz, 1H), 4.22 (d, J=10 Hz, 1H), 3.98 (m, 1H), 3.88 (m, 1H), 3.65 (s, 6H), 3.40 (d, J=15 Hz, 1H), 3.04 (d, J=20 Hz, 1H), 2.28 (m, 3H), 2.19 (m, 1H), 2.03 (m, 3H), 1.93 (m, 1H), 1.36 (d, J=15 Hz, 1H), 1.24 (m, 1H), 1.08 (d, J=10 Hz, 1H), 1.02 (d, J=10 Hz, 1H), 0.93-0.89 (m, 12H), 0.55 (m, 2H), 0.35 (s, 3H), 0.29 (s, 3H), 0.24 (m, 2H). LC/MS: Anal. Calcd. For [(M+2H)/2]⁺ C51H60FN9O7SSi: 495.7; found 496.0. Separation method used: Xbridge C18 5 um 19×50 mm, 25 mL/min, 25 C, 17% ACN (0.1% NH₄OH) to 90% over 8 min, UV 215 nm.

Compounds 14-19, depicted in the table below, were made using the methods described in Example 10 and substituting the appropriate reactants and/or reagents.

| | | Ob- |
|---|---|---|
| Com- | | served |
| pound | | [(M + |
| ID | Structure | 2H)/2]⁺ |
| 14 | 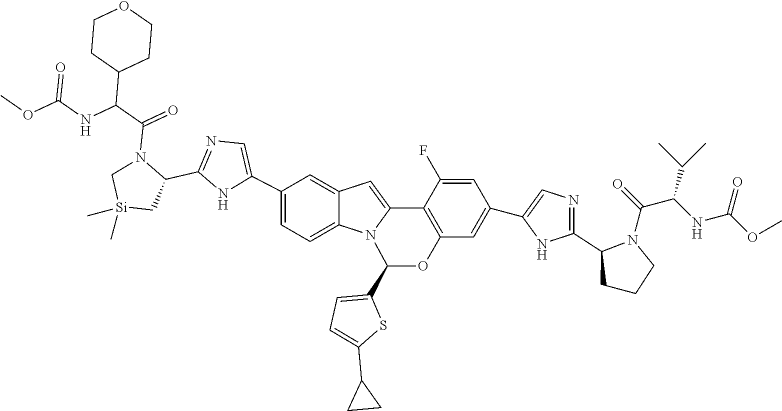 | 517.1 |
| 15 | 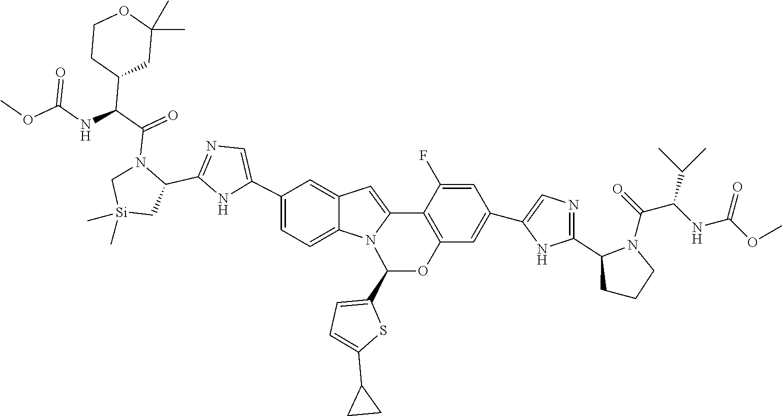 | 530.7 |
| 16 | 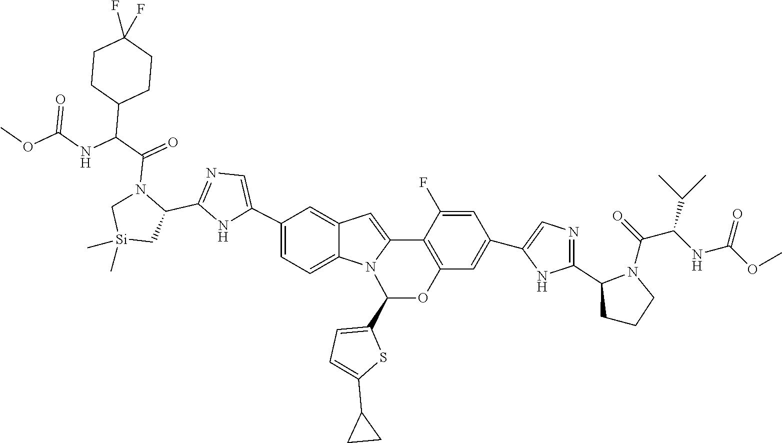 | 534.1 |
| 17 | 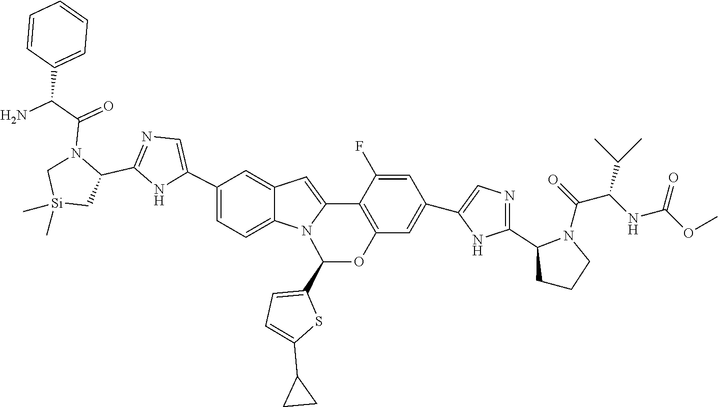 [SMILES: COC(=O)N[C@H](C(=O)N1CCC[C@H]1c1ncc(-c2cc(F)c3c(c2)O[C@@H](c2ccc(C4CC4)s2)n2c-3cc3cc(-c4cnc([C@@H]5C[Si](C)(C)CN5C(=O)[C@H](N)c5ccccc5)[nH]4)ccc32)[nH]1)C(C)C] | 483.6 |
| 18 | 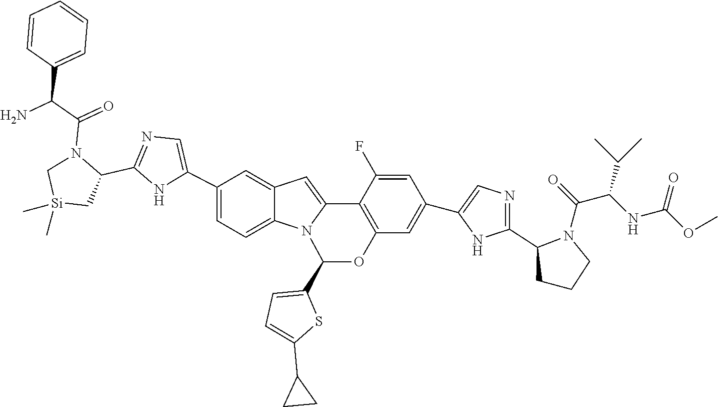 [SMILES: COC(=O)N[C@H](C(=O)N1CCC[C@H]1c1ncc(-c2cc(F)c3c(c2)O[C@@H](c2ccc(C4CC4)s2)n2c-3cc3cc(-c4cnc([C@@H]5C[Si](C)(C)CN5C(=O)[C@@H](N)c5ccccc5)[nH]4)ccc32)[nH]1)C(C)C] | 483.6 |
| 19 | 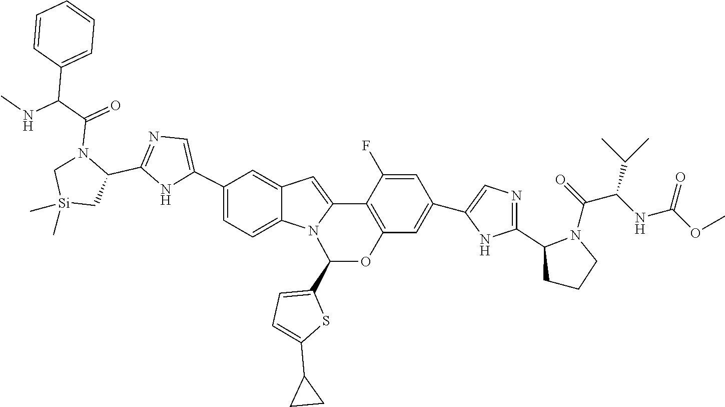 | 491.0 |

### Example 11

### Cell-Based HCV Replicon Assays

To measure cell-based anti-HCV activity of compounds of the present invention, two complimentary assays were employed using various replicons. In the first assay (“Replicon Assay A”), replicon cells were seeded at 2000 cells/well in 384-well 384-well flat bottom tissue culture treated clear bottom plate (Corning 3707) in the presence of the test compound. Various concentrations of test compound, typically in 10 serial dilutions, were added to the assay mixture, with the starting concentration ranging from 333.3 nM to 1.667 nM. The final concentration of DMSO was 0.5%. Fetal bovine serum was 5%, in the assay media. Cells were harvested on day 3 by removing media and washing the cells with a suitable wash buffer. The cells are lysed with the addition of 1× Qiagen lysis buffer (Cat #1062731). The replicon RNA level was measured using real time PCR (TaqMan® EZ RT-PCR, Applied Biosystems 403028) with the following primers and probes:

Neo Forward:
CCG GCT ACC TGC CCA TTC
Neo Reverse:
CCA GAT CAT CCT GAT CGA CAA G
Neo Probe:
FAM-ACA TCG CAT CGA GCG AGC ACG TAC-Tamra
Cyc probe:
5′-JOE-CGCGTCTCCTTTGAGCTGTTTGCA-Tamra-3′
Cyc Forward Primer:
ACGGCGAGCCCTTGG
Cyc Reverse Primer:
TTTCTGCTGTCTTTGGGACCT

Cyclophilin RNA was used as endogenous control and was amplified in the same reaction as NS5B (multiplex PCR). The real-time RT-PCR reactions were run on ABI PRISM 7900HT Sequence Detection System using the following program: 50° C. for 2 minutes, 60° C. for 30 minutes, 95° C. for 5 minutes, 40 cycles of 94 C for 20 sec, 55 C for 1 minutes.

The amount of HCV replicon RNA per cell is quantified using a linear regression curve for a known nanogram (ng) amount of HCV replicon total RNA. This is established by plotting the Cycle Threshold values (Ct) from the Neo probe and primer set versus the log (ng) for each HCV replicon total RNA standard. The amount of HCV RNA for each replicon sample is calculated by taking the sample's Ct value, minus the line intercept, divided by the slope of the line. Similarly, the amount of Cyclophilin mRNA per cell is also quantified using a linear regression curve for a known nanogram (ng) amount of HCV replicon total RNA. Again, this is established by plotting the Cycle Threshold values (Ct) from the Cyclophilin probe and primer set versus the log (ng) for each HCV replicon total RNA standard.

In an alternate assay (“Replicon Assay B”), 1000 cells were seeded per well in a 384-well collagen coated black plate from Greiner bio-one (Cat #781946) in 5% FBS. Inhibitors of this invention were added at 24 h post-seeding, and the plates were incubated for 3 days. Cells were subsequently lysed with Qiagen lysis buffer (Cat #1062731) to extract the RNA. HCV replicon RNA level was measured by real-time PCR using the RNA-to-CT kit from Applied Biosystem (Cat #4392656) and genotype-specific primers and probes. The amplicon was located within NS5B. The sequence of the PCR primers are as follows: 5B.2F, ATGGACAGGCGCCCTGA (SEQ. ID NO. 1); 5B.2R, TTGATGGGCAGCTTGGTTTC (SEQ. ID NO. 2); the probe sequence was FAM-labeled CACGCCATGCGCTGCGG (SEQ. ID NO. 3). To detect genotype 1A the primer 1A F, TGCGGAACCGGTGAGTACA and 1A R, GCGGGTTTATCCAAGAAAGGA were used; the probe sequence was FAM-CGGAATTGCCAGGACGACCGG.

The real-time RT-PCR reactions were run on ABI PRISM 7900HT or Viia7 Sequence Detection System using the following program: 48° C. for 30 minutes, 95 C for 10 minutes, 40 cycles of 95 C for 15 sec, 60 C for 1 minutes. The 50% effective concentration (EC₅₀) was the drug concentration necessary to achieve an increase in the cycle threshold (C_{T}) of 1 over the projected baseline C_{T}. The EC₉₀ was the drug concentration necessary to achieve an increase in C_{T} of 3.2 over the projected baseline C_{T}.

Data was obtained for the compounds of the present invention against various replicons using the methods described in the Example above, and is presented in the tables immediately below.

| | Gt1a_ | GT1a_ | GT1a_ | GT1a_ | GT1a_ |
|---|---|---|---|---|---|
| | WT | M28V | Q30R | L31V | Y93H |
| Compound | EC90 | EC90 | EC90 | EC90 | EC90 |
| ID | (nM) | (nM) | (nM) | (nM) | (nM) |
| 1 | 0.00042 | 0.00071 | 0.0193 | 0.0568 | 0.231 |
| 2 | 0.00028 | 0.00027 | 0.0043 | 0.0030 | 0.062 |
| 3 | 0.00066 | 0.00050 | 0.0080 | 0.0097 | 0.079 |
| 4 | 0.00085 | 0.00057 | 0.0311 | 0.0522 | 0.351 |
| 5 | 0.00055 | 0.00062 | 0.0155 | 0.0359 | 0.330 |
| 6 | 0.00051 | 0.00047 | 0.0729 | 0.0599 | 0.199 |
| 7 | 0.00062 | 0.00061 | 0.0039 | 0.0045 | 0.022 |
| 8 | 0.00029 | 0.00041 | 0.0056 | 0.0088 | 0.271 |
| 9 | 0.00052 | 0.00042 | 0.0247 | 0.0260 | 0.290 |
| 10 | 0.00047 | 0.00063 | 0.3874 | 0.8593 | 4.280 |
| 11 | 0.00036 | 0.00043 | 0.0318 | 0.0963 | 0.933 |
| 12 | 0.00046 | 0.00051 | 0.0776 | 0.1492 | 0.334 |
| 13 | 0.00048 | 0.00045 | 0.0328 | 0.0756 | 0.561 |
| 14 | 0.00058 | 0.00058 | 0.0027 | 0.0062 | 0.020 |
| 15 | 0.00060 | 0.00076 | 0.0047 | 0.0075 | 0.185 |
| 16 | 0.00085 | 0.00064 | 0.0285 | 0.0507 | 0.972 |
| 17 | 0.00142 | 0.00192 | 0.8960 | 1.7570 | 6.183 |
| 18 | 0.00055 | 0.00071 | 0.0372 | 0.0681 | 1.223 |
| 19 | 0.00035 | 0.00039 | 0.0053 | 0.0106 | 0.219 |
| | GT1b_ | | GT2b_ | GT3a_ | GT3a_ |
| | WT | GT2a | 31M | WT | Y93H |
| Compound | EC90 | EC90 | EC90 | EC90 | EC90 |
| ID | (nM) | (nM) | (nM) | (nM) | (nM) |
| 1 | 0.00070 | 0.00278 | 2.430 | 1.767 | 393 |
| 2 | 0.00059 | 0.00049 | 0.231 | 0.170 | 168 |
| 3 | 0.00100 | 0.00167 | 0.431 | 0.285 | 370 |
| 4 | 0.00160 | 0.00393 | 0.448 | 0.169 | 587 |
| 5 | 0.00210 | 0.00095 | 0.378 | 0.257 | 110 |
| 6 | 0.00116 | 0.00339 | 3.038 | 1.322 | 118 |
| 7 | 0.00244 | 0.00177 | 0.132 | 0.051 | 82 |
| 8 | 0.00096 | 0.00118 | 1.117 | 0.709 | 133 |
| 9 | 0.00108 | 0.00243 | 4.235 | 2.238 | 322 |
| 10 | 0.00142 | 0.00246 | 12.015 | 5.478 | 126 |
| 11 | 0.00154 | 0.00100 | 0.705 | 0.347 | 74 |
| 12 | 0.00141 | 0.00152 | 0.061 | 0.036 | 155 |
| 13 | 0.00118 | 0.00338 | 0.933 | 0.469 | 614 |
| 14 | 0.00131 | 0.00420 | 0.038 | 0.018 | 175 |
| 15 | 0.00102 | 0.00210 | 0.314 | 0.173 | 442 |
| 16 | 0.00184 | 0.00575 | 2.830 | 1.681 | 3690 |
| 17 | 0.00141 | 0.05820 | 5.325 | 3.278 | 765 |
| 18 | 0.00111 | 0.00085 | 0.062 | 0.027 | 206 |
| 19 | 0.00081 | 0.00062 | 0.092 | 0.056 | 109 |

| | GT4 | GT5 | GT6 |
|---|---|---|---|
| Compound | EC90 | EC90 | EC90 |
| ID | (nM) | (nM) | (nM) |
| 1 | 0.00086 | 0.0056 | 0.0059 |
| 2 | 0.00071 | 0.0041 | 0.0044 |
| 3 | 0.00112 | 0.0073 | 0.0076 |
| 4 | 0.00135 | 0.0227 | 0.1670 |
| 5 | 0.00088 | 0.0086 | 0.0088 |
| 6 | 0.00135 | 0.0069 | 0.0057 |
| 7 | 0.00147 | 0.0246 | 0.0906 |
| 8 | 0.00103 | 0.0072 | 0.0079 |
| 9 | 0.00137 | 0.0090 | 0.0109 |
| 10 | 0.00096 | 0.0139 | 0.0080 |
| 11 | 0.00081 | 0.0098 | 0.0066 |
| 12 | 0.00089 | 0.0144 | 0.0863 |
| 13 | 0.00150 | 0.0061 | 0.0108 |
| 14 | 0.00111 | 0.0130 | 0.0842 |
| 15 | 0.00136 | 0.0065 | 0.0144 |
| 16 | 0.00183 | 0.0173 | 0.0252 |
| 17 | 0.01241 | 0.0527 | 0.1051 |
| 18 | 0.00092 | 0.0068 | 0.0084 |
| 19 | 0.00071 | 0.0048 | 0.0047 |

1. A compound having the formula (I): 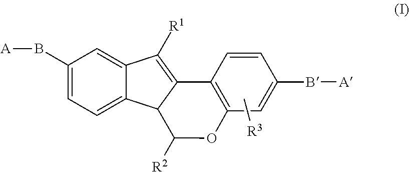
or a pharmaceutically acceptable salt thereof, wherein:
is selected from 4 to 7-membered monocyclic heterocycloalkyl or R¹¹, wherein said 4 to 7-membered monocyclic heterocycloalkyl group and said R¹¹ group is substituted on a ring nitrogen atoms with R⁴, and optionally further substituted on one or more ring carbon atoms with R⁵, such that two R⁵ groups on the same ring carbon atom, together with the carbon atom to which they are attached, can join to form a spirocyclic C₃-C₇ cycloalkyl group or a spirocyclic 4 to 7-membered monocyclic heterocycloalkyl group; or two R⁵ groups attached to the same A ring, together with the carbon atoms to which they are attached, can join to form a fused C₃-C₇ cycloalkyl group, a bridged C₃-C₇ cycloalkyl group or a fused 4 to 7-membered monocyclic heterocycloalkyl group;
A′ is selected from 4 to 7-membered monocyclic heterocycloalkyl or R¹¹, wherein said 4 to 7-membered monocyclic heterocycloalkyl group and said R¹¹ group is substituted on a ring nitrogen atoms with R⁴, and optionally further substituted on one or more ring carbon atoms with R⁵, such that two R⁵ groups on the same ring carbon atom, together with the carbon atom to which they are attached, can join to form a spirocyclic C₃-C₇ cycloalkyl group or a spirocyclic 4 to 7-membered monocyclic heterocycloalkyl group; or
two R⁵ groups attached to the same A ring, together with the carbon atoms to which they are attached, can join to form a fused C₃-C₇ cycloalkyl group, a bridged C₃-C₇ cycloalkyl group or a fused 4 to 7-membered monocyclic heterocycloalkyl group;
B is: 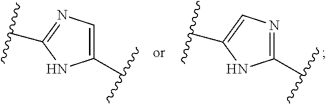 [SMILES: *c1cnc(*)[nH]1 ; *c1cnc(*)[nH]1]
B′ is: 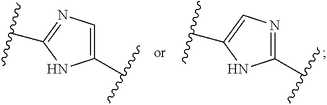 [SMILES: *c1cnc(*)[nH]1 ; *c1cnc(*)[nH]1]
R¹ is selected from H, C₁-C₆ alkyl, C₃-C₇ cycloalkyl, 4 to 7-membered heteroaryl, phenyl and halo;
R² is selected from 5 or 6-membered monocyclic heteroaryl and 9 or 10-membered bicyclic heteroaryl, wherein said 5 or 6-membered monocyclic heteroaryl group and said 9 or 10-membered bicyclic heteroaryl group each can be optionally substituted on one or more ring carbon atoms with R⁶;
R³ represents up to 3 optional phenyl group substituents, each independently selected from halo, —CN, C₁-C₆ alkyl, C₁-C₆ haloalkyl, C₃-C₇ cycloalkyl, 4 to 6-membered monocyclic heterocycloalkyl, 5 or 6-membered monocyclic heteroaryl, C₆-C₁₀ aryl, benzyl and —O—(C₁-C₆ alkyl), wherein said C₃-C₇ cycloalkyl group, said 4 to 6-membered monocyclic heterocycloalkyl group, said 5 or 6-membered monocyclic heteroaryl group, said C₆-C₁₀ aryl group, or the phenyl moiety of said benzyl group can be optionally substituted with up to 3 groups, which can be the same or different, and are selected from halo, —CN, C₁-C₆ alkyl, C₁-C₆ haloalkyl, —O—C₁-C₆ alkyl, —(C₁-C₆ alkylene)-O—C₁-C₆ alkyl and —O—(C₁-C₆ haloalkyl);
each occurrence of R⁴ is independently: 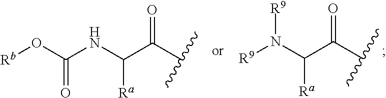
R^{a} is selected from C₁-C₆ alkyl, C₁-C₆ haloalkyl, C₁-C₆ silylalkyl, C₃-C₇ cycloalkyl, 4- to 7-membered monocyclic heterocycloalkyl, phenyl and 5 or 6-membered monocyclic heteroaryl, wherein said C₃-C₇ cycloalkyl group, said 4 to 7-membered monocyclic heterocycloalkyl group, said phenyl group, and said 5 or 6-membered monocyclic heteroaryl group can each be optionally substituted on one or more ring carbon atoms with R⁶;
R^{b} is selected from H, C₁-C₆ alkyl, C₁-C₆ haloalkyl, C₁-C₆ silylalkyl, C₃-C₇ cycloalkyl, 4- to 7-membered monocyclic heterocycloalkyl, phenyl and 5 or 6-membered monocyclic heteroaryl;
each occurrence of R⁵ is independently selected from C₁-C₆ alkyl, C₁-C₆ haloalkyl, C₃-C₇ cycloalkyl, 4 to 7-membered monocyclic heterocycloalkyl, phenyl, 5 or 6-membered monocyclic heteroaryl, halo, —CN, —OR⁸, —N(R⁷)₂, —C(O)R¹⁰, —C(O)OR⁸, —C(O)N(R⁸)₂, —NHC(O)R⁸, —NHC(O)NHR⁸, —NHC(O)OR⁸, —OC(O)R⁸, —SR⁸, —S(O)₂R⁸ and Si(R¹⁰)₃, wherein said C₃-C₇ cycloalkyl group, said 4 to 7-membered monocyclic heterocycloalkyl group, said phenyl group, and said 5 or 6-membered monocyclic heteroaryl group can each be optionally substituted on one or more ring carbon atoms with R⁶;
each occurrence of R⁶ is independently selected from halo, —CN, C₁-C₆ alkyl, C₃-C₇ cycloalkyl, C₁-C₆ haloalkyl, —O—(C₁-C₆ haloalkyl), C₂-C₆ alkynyl, C₁-C₆ hydroxyalkyl, —(C₁-C₆ alkylene)ₘ-O—(C₁-C₆ alkyl), —N(R⁷)₂, C₆-C₁₀ aryl, —(C₁-C₆ alkylene)ₘ-(C₃-C₇ cycloalkyl), —O—(C₆-C₁₀ aryl), 4 to 7-membered monocyclic heterocycloalkyl, 5 or 6-membered monocyclic heteroaryl, —O-(5 or 6-membered monocyclic heteroaryl), 8 to 10-membered bicyclic heteroaryl and —O-(8 to 10-membered bicyclic heteroaryl), wherein said C₆-C₁₀ aryl group, said C₃-C₇ cycloalkyl group, said 4 to 7-membered monocyclic heterocycloalkyl group, said 5 or 6-membered monocyclic heteroaryl group and said 8 to 10-membered bicyclic heteroaryl group can be optionally substituted with up to 3 groups, each independently selected from halo, hydroxy, C₁-C₆ alkyl, C₁-C₆ haloalkyl and —O—(C₁-C₆ alkyl), and wherein said C₆-C₁₀ aryl group, said 5 or 6-membered monocyclic heteroaryl group and said 8 to 10-membered bicyclic heteroaryl group, can be optionally fused with a C₃-C₆ cycloalkyl group;
each occurrence of R⁷ is independently selected from H, C₁-C₆ alkyl, or C₃-C₇ cycloalkyl;
each occurrence of R⁸ is independently selected from H, C₁-C₆ alkyl, C₁-C₆ haloalkyl, —C₁-C₆ alkylene-OC(O)(C₁-C₆ alkyl), C₁-C₆ hydroxyalkyl, C₃-C₇ cycloalkyl, 4 to 7-membered monocyclic heterocycloalkyl, phenyl and 5 or 6-membered monocyclic heteroaryl, wherein said C₃-C₇ cycloalkyl group, said 4- to 7-membered monocyclic heterocycloalkyl group, said phenyl group and said 5 or 6-membered monocyclic heteroaryl group can be optionally and independently substituted with up to three groups independently selected from —OH, halo, C₁-C₆ alkyl, C₁-C₆ haloalkyl, —NH(C₁-C₆ alkyl) and —N(C₁-C₆ alkyl)₂;
each occurrence of R⁹ is independently selected from H, C₁-C₆ alkyl, or C₃-C₇ cycloalkyl, or two R⁹ groups, together with the common N to which they are attached, can join to form a 3 to 7 membered heterocycloalkyl group;
each occurrence of R¹⁰ is independently selected from C₁-C₆ alkyl, C₃-C₇ cycloalkyl, 4- to 7-membered monocyclic heterocycloalkyl, phenyl, 5 or 6-membered monocyclic heteroaryl, C₁-C₆ haloalkyl, —CN and —OR³, wherein said C₃-C₇ cycloalkyl group, said 4 to 7-membered monocyclic heterocycloalkyl group, said phenyl group, and said 5 or 6-membered monocyclic heteroaryl group can each be optionally substituted on one or more ring carbon atoms with R⁶, or optionally, two R¹⁰ groups, together with the common silicon atom to which they are attached, can optionally join to form a 4- to 7-membered silyl-containing monocyclic heterocycloalkyl ring; each occurrence of R¹¹ is independently selected from monocyclic 5- to 7-membered silylheterocycloalkyl ring and a bicyclic 7- to 11-membered silylheterocycloalkyl ring wherein said silylheterocycloalkyl rings contain as heteroatom ring members:
(i) one —Si(R¹⁰)₂—;
(ii) one —N(R⁴)—; and
(iii) one optional and additional heteroatom ring member selected from the group consisting of nitrogen, oxygen and sulfur,
and wherein an R¹¹ group can be optionally and independently substituted on one or two ring carbon atoms with R⁵; and
each occurrence of m is independently 0 or 1; wherein at least one of A and A′ is R¹¹.

2. The compound of claim 1, wherein B is: 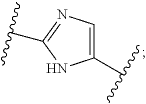 [SMILES: *c1cnc(*)[nH]1]
and B′ is: 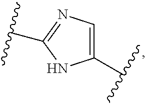 [SMILES: *c1cnc(*)[nH]1]
or a pharmaceutically acceptable salt thereof.

3. The compound of claim 1 wherein one of A and A′ is R¹¹ and the other of A and A′ is 4 to 7-membered monocyclic heterocycloalkyl, or a pharmaceutically acceptable salt thereof.

4. The compound of claim 3, wherein one of A and A′ is selected from; 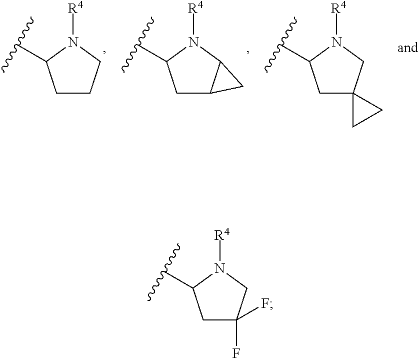
and the other of A and A′ is selected from: 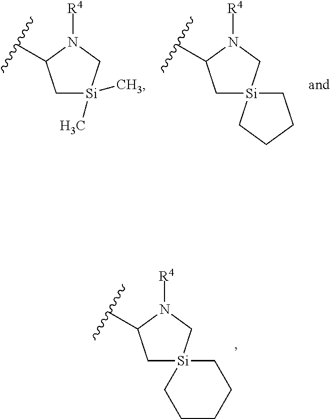
or a pharmaceutically acceptable salt thereof.

5. The compound of claim 1, wherein each occurrence of R⁴ is: 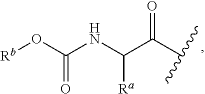
wherein
each occurrence of R^{a} is independently selected from C₁-C₆ alkyl, C₃-C₇ cycloalkyl, 4- to 7-membered monocyclic heterocycloalkyl and phenyl, wherein said 4- to 7-membered monocyclic heterocycloalkyl group can be optionally substituted with up to 4 substituents, each of which are independently selected from halo and C₁-C₆ alkyl; and
each occurrence of R^{b} is independently selected from C₁-C₆ alkyl;
or a pharmaceutically acceptable salt thereof.

6. The compound of claim 1, wherein each occurrence of R⁴ is:

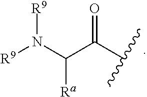

7. The compound of claim 1, having the formula (Ia): 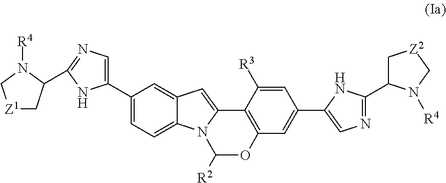
wherein:
R² is 5-membered heteroaryl, which can be optionally substituted with C1-C6 alkyl or C3-C7 cycloalkyl;
R³ is H or halo;
each occurrence of R⁴ is: 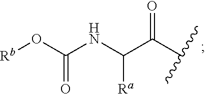
each occurrence of R^{a} is independently selected from C₁-C₆ alkyl, C₃-C₆ cycloalkyl, 4- to 7-membered monocyclic heterocycloalkyl and phenyl, wherein said 4- to 7-membered monocyclic heterocycloalkyl group can be optionally substituted with up to 4 substituents, each of which are independently selected from halo and C₁-C₆ alkyl;
each occurrence of R^{b} is independently selected from C₁-C₆ alkyl;
Z¹ is —Si(R¹⁰)₂— or —C(R⁵)₂—;
Z² is —Si(R¹⁰)₂— or —C(R⁵)₂—;
each occurrence of R⁵ is independently H or F, or or two R⁵ groups that are attached to the same carbon atom, combine to form a spirocyclic C₃-C₅ cycloalkyl group;
each occurrence of R¹⁰ is independently C₁-C₆ alkyl, or two R¹⁰ groups that are attached to the same Si atom, combine to form a —(CH₂)₄— or —(CH₂)₅— group; and
such that at least one of Z¹ and Z² is Si(R¹⁰)₂,
or a pharmaceutically acceptable salt thereof.

8. The compound of claim 1, wherein each occurrence of R⁴ is: 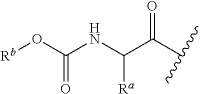
wherein R^{a} is isopropyl, cyclopropyl, phenyl, 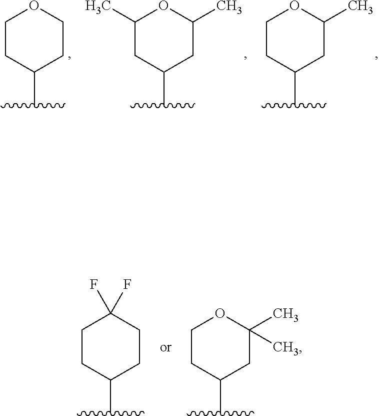
or a pharmaceutically acceptable salt thereof.

9. The compound of claim 8, wherein each occurrence of R⁴ is independently selected from: 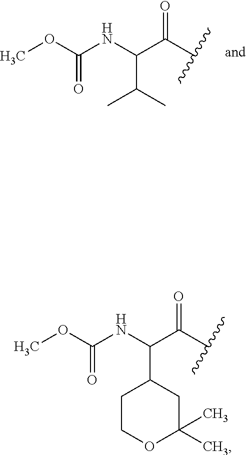
or a pharmaceutically acceptable salt thereof.

10. The compound of claim 7, wherein Z¹ is —Si(R¹⁰)₂—, or a pharmaceutically acceptable salt thereof.

11. The compound of claim 7, wherein Z² is —Si(R¹⁰)₂—, or a pharmaceutically acceptable salt thereof.

12. The compound of claim 7, wherein Z¹ is CH₂, —CH(F) or —CF₂—, or a pharmaceutically acceptable salt thereof.

13. The compound of claim 7, wherein Z² is CH₂, —CH(F) or —CF₂—, or a pharmaceutically acceptable salt thereof.

14. The compound of claim 1 being any one of the compounds numbered 1 to 25 in the above specification, or a pharmaceutically acceptable salt thereof.

15. A pharmaceutical composition comprising an effective amount of a compound of claim 1, or a pharmaceutically acceptable salt thereof, and a pharmaceutically acceptable carrier.

16. The pharmaceutical composition of claim 15, further comprising a second therapeutic agent selected from the group consisting of HCV antiviral agents, immunomodulators, and anti-infective agents.

17. The pharmaceutical composition of claim 16, further comprising a third therapeutic agent selected from the group consisting of HCV protease inhibitors, HCV NS5A inhibitors and HCV NS5B polymerase inhibitors.

18. (canceled)

19. A method of treating a patient infected with HCV comprising the administering to said patient a compound of claim 1, or a pharmaceutically acceptable salt thereof, in an amount effective to prevent and/or treat infection by HCV in said patient.

20. The method of claim 19, further comprising administering one or more additional therapeutic agents to said patient, wherein said additional therapeutic agents are selected from the group consisting of HCV protease inhibitors and HCV NS5B polymerase inhibitors.

21. The composition of claim 17, wherein said HCV NS5B polymerase inhibitor is a nucleoside compound.

22. (canceled)
